(19) United States
(12) Patent Application Publication
(10) Pub. No.: US 2020/0234226 A1
(43) Pub. Date: Jul. 23, 2020

(54) SYSTEM FOR MANAGEMENT BY OBJECTIVES, SERVER FOR MANAGEMENT BY OBJECTIVES, PROGRAM FOR MANAGEMENT BY OBJECTIVES, AND TERMINAL DEVICE FOR MANAGEMENT BY OBJECTIVES

(71) Applicants: OMRON HEALTHCARE Co., Ltd., Muko-shi (JP); OMRON Corporation, Kyoto-shi (JP)

(72) Inventors: Sho NAGAYOSHI, Kyoto (JP); Hiroshi KOSHIMIZU, Kyoto (JP); Ken MIYAGAWA, Kyoto (JP); Keiichi OBAYASHI, Tokyo (JP)

(73) Assignees: OMRON HEALTHCARE Co., Ltd., Muko-shi (JP); OMRON Corporation, Kyoto-shi (JP)

(21) Appl. No.: 16/843,240

(22) Filed: Apr. 8, 2020

Related U.S. Application Data
(63) Continuation of application No. PCT/JP2018/039020, filed on Oct. 19, 2018.

(30) Foreign Application Priority Data
Oct. 26, 2017 (JP) 2017-207225

(51) Int. Cl. G06Q 10/06; G06N 5/02

(52) U.S. Cl. CPC G06Q 10/063118; G06N 5/02

(57) ABSTRACT

A storage unit stores a value indicating a change in a value for a body with respect to an action in type or amount and uses the value stored therein and indicating the change to predict a value indicating a change in a value for the body of a user for each action of the user, and presents the value indicating the change as predicted. A value indicating a change arising when a prescribed action is performed and a value indicating a change arising when the prescribed action is not performed are predicted. The storage unit stores a value indicating how the value for the body of the user changes with respect to the user's action in type or amount. The storage unit stores values indicating how values for bodies of a plurality of people change with respect to actions of the plurality of people in type or amount.

The present application is a continuation of International application No. PCT/JP2018/039020, filed Oct. 19, 2018, which claims priority to Japanese Patent Application No. 2017-207225, filed Oct. 26, 2017, the entire contents of each of which are incorporated herein by reference.

### BACKGROUND OF THE INVENTION

### Field of the Invention

The present disclosure relates to a system for management by objectives, a server for management by objectives, a program for management by objectives, and a terminal device for management by objectives. In particular, the present disclosure relates to a system for management by objectives, a server for management by objectives, a program for management by objectives, and a terminal device for management by objectives suitable for management by objectives for an objective for a user's body.

### Description of the Background Art

Conventionally, there has been a system for management by objectives for a user's body. In such a system, the current value of an indicator of the body composition of a subject is obtained, an average amount of a change of the value of the indicator from the subject's current age to an age in the future is calculated based a regression equation with age serving as an independent variable, and a value of the indicator at the age of the subject in the future is determined as a future value based on the current value and the amount of the change (for example, see Japanese Patent Laying-Open No. 2013-81800 (hereinafter referred to as “Patent Literature 1”).

### CITATION LIST

### Patent Literature

PTL 1: Japanese Patent Laying-Open No. 2013-81800

### SUMMARY OF THE INVENTION

### Technical Problem

A value for a user's body changes with the user's action. However, the system of Patent Literature 1 does not consider such an action of the user. For this reason, depending on the action of the user, there is a possibility that an error occurs in predicting how a value for the user's body will change in the future.

An object in an aspect of the present disclosure is to provide a system for management by objectives, a server for management by objectives, a program for management by objectives, and a terminal device for management by objectives that can predict how a value for a user's body changes for each action of the user.

### Solution to Problem

In one aspect of the present disclosure a system for management by objectives is a system that performs management by objectives for a body of a user, and comprises a storage unit, a prediction unit and a presentation unit. The storage unit previously stores a value indicating a change in a value for a body with respect to an action in type or amount. The prediction unit uses the value stored in the storage unit and indicating the change to predict a value indicating a change in a value for the body of the user for each action of the user. The presentation unit presents the value indicating the change predicted by the prediction unit.

Preferably, the prediction unit predicts a value indicating a change arising when a prescribed action is performed and a value indicating a change arising when the prescribed action is not performed.

Preferably, the storage unit previously stores a value indicating how the value for the body of the user changes with respect to the user's action in type or amount.

Preferably, the storage unit previously stores values indicating how values for bodies of a plurality of people change with respect to actions of the plurality of people in type or amount.

Preferably, the system for management by objectives further comprises a server and a terminal device. The server includes the storage unit and the prediction unit. The terminal device includes the presentation unit.

In another aspect of the present disclosure a server for management by objectives is a server that performs management by objectives for a body of a user, and comprises a storage unit, a prediction unit and a presentation unit. The storage unit previously stores a value indicating a change in a value for a body with respect to an action in type or amount. The prediction unit uses the value stored in the storage unit and indicating the change to predict a value indicating a change in a value for the body of the user for each action of the user. The transmission unit transmits the value predicted by the prediction unit and indicating the change to the terminal device for presentation via the terminal device.

In still another aspect of the present disclosure a program for management by objectives is executed in a server that performs management by objectives for a body of a user. The server includes a storage unit that previously stores a value indicating a change in a value for a body with respect to an action in type or amount. The program for management by objectives causes the server to: use the value stored in the storage unit and indicating the change to predict a value indicating a change in a value for the body of the user for each action of the user; and transmit the value indicating the predicted change to the terminal device for presentation via the terminal device.

In still another aspect of the present disclosure a terminal device for management by objectives is a terminal device that performs management by objectives for a body of a user, and comprises a reception unit and a presentation unit. The reception unit receives a value indicating a change in a value for the body of the user for each action of the user that is predicted by a server using a value previously stored in the server and indicating a change in a value for a body with respect to an action in type or amount. The presentation unit presents the value indicating the change received by the reception unit.

In still another aspect of the present disclosure a program for management by objectives is executed in a terminal device that performs management by objectives for a body of a user. The program causes the terminal device to: receive a value indicating a change in a value for the body of the user for each action of the user that is predicted by a server using a value previously stored in the server and indicating a change in a value for a body with respect to an action in type or amount; and present the received value indicating the change.

### Advantageous Effects of Invention

According to the present disclosure, there can be provided a system for management by objectives, a server for management by objectives, a program for management by objectives, and a terminal device for management by objectives capable of predicting how a value for a user's body changes for each action of the user.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 shows a step 1 for improving a habit.

FIG. 2 shows a step 2 for improving the habit.

FIG. 3 shows a step 3 for improving the habit.

FIG. 4 shows a step 4 for improving the habit.

FIG. 5 shows a step 5 for improving the habit.

FIG. 6 shows a step 6 for improving the habit.

FIG. 7 shows a step 7 for improving the habit.

FIG. 8 shows a step 8 for improving the habit.

FIG. 9 shows a step 9 for improving the habit.

FIG. 10 is a diagram schematically illustrating a general configuration of a system for management by objectives according to an embodiment.

FIG. 11 is a block diagram illustrating a configuration of an information communication terminal according to the present embodiment.

FIG. 12 is a block diagram illustrating a configuration of a server for management by objectives according to the present embodiment.

FIG. 13 is a block diagram illustrating a configuration of a biological information measuring device according to the present embodiment.

FIG. 14 is a flowchart of a management by objectives process performed by the server for management by objectives according to the present embodiment.

FIG. 15 is a flowchart of a first half of an objective realization process performed by the server for management by objectives according to the present embodiment.

FIG. 16 is a diagram illustrating an example of a morphological analysis according to the present embodiment.

FIG. 17 is a diagram illustrating an example of classification of an objective by category according to the present embodiment.

FIG. 18 is a diagram illustrating an example of a feature representation space according to the present embodiment.

FIG. 19 is a first diagram illustrating an example of a process of creating a feature representation map according to the present embodiment.

FIG. 20 is a second diagram illustrating an example of a process of creating a feature representation map according to the present embodiment.

FIG. 21 shows an example of a database at an initial stage of a value of an indicator for a body composition of a “cool” person according to the present embodiment.

FIG. 22 shows an example of a database after data is accumulated for a value for an indicator for a “cool” person's body composition according to the present embodiment.

FIG. 23 shows an example of a language database of categories for a body composition according to the present embodiment.

FIG. 24 is a first diagram illustrating an example of a process of specifying a quantitative objective value from a feature representation map according to the present embodiment.

FIG. 25 is a second diagram illustrating an example of a process of specifying a quantitative objective value from a feature representation map according to the present embodiment.

FIG. 26 is a third diagram illustrating an example of a process of specifying a quantitative objective value from a feature representation map according to the present embodiment.

FIG. 27 is a diagram for illustrating presentation of a quantitative objective value according to the present embodiment.

FIG. 28 shows an example of a display screen displayed at a display unit of an information communication terminal in the first half of the objective realization process according to the present embodiment.

FIG. 29 is a flowchart of a second half of the objective realization process performed by the server for management by objectives according to the present embodiment.

FIG. 30 shows an example of a process of obtaining information of when an objective is achieved according to the present embodiment.

FIG. 31 is a diagram illustrating an example of a semantic analysis of a word designating a time according to the present embodiment.

FIG. 32 is a diagram showing a gap with an objective value according to the present embodiment.

FIG. 33 is a diagram illustrating an example of a route to achieve an objective according to the present embodiment.

FIG. 34 is a diagram illustrating an example of a process of determining a recommended route to achieve an objective according to the present embodiment.

FIG. 35 shows an example of a display screen displayed at a display unit of an information communication terminal in the second half of the objective realization process according to the present embodiment.

FIG. 36 shows an example of a process of selecting a method for achieving an objective according to the present embodiment.

FIG. 37 is a flowchart of a management by objectives progress management process performed by the server for management by objectives in the present embodiment.

FIG. 38 shows an example of a track record of a group for effectiveness of intervention according to the present embodiment.

FIG. 39 is a diagram for illustrating a process of calculating a method for intervention by using a track record of a group according to the present embodiment.

FIG. 40 is a first diagram for illustrating a process of calculating an intervention threshold value by using a track record of a group according to the present embodiment.

FIG. 41 is a second diagram for illustrating the process of calculating the intervention threshold value by using the track record of the group according to the present embodiment.

FIG. 42 shows an example of a track record of an individual for effectiveness of intervention according to the present embodiment.

FIG. 43 is a diagram for illustrating a process of calculating a method for intervention by using a track record of an individual according to the present embodiment.

FIG. 44 is a first diagram for illustrating a process of calculating an intervention threshold value by using a track record of an individual according to the present embodiment.

FIG. 45 is a second diagram for illustrating the process of calculating the intervention threshold value by using the track record of the individual according to the present embodiment.

FIG. 46 is a diagram for illustrating a degree of progress of management by objectives according to the present embodiment.

FIG. 47 is a flowchart of an objective maintenance process performed by the server for management by objectives according to the present embodiment.

FIG. 48 shows past data of a group that is extracted as the data is similar to that of a user of interest according to the present embodiment.

FIG. 49 represents a predicted transition of a change in an indicator of data similar to that of the user of interest according to the present embodiment.

FIG. 50 shows an individual's past data extracted according to the present embodiment.

FIG. 51 is a diagram illustrating a predicted transition of a change in an indicator for the user of interest according to the present embodiment.

FIG. 52 shows an error evaluation using a group prediction model according to the present embodiment.

FIG. 53 shows an error evaluation using an individual prediction model according to the present embodiment.

FIG. 54 shows a result of predicting how an indicator changes according to the present embodiment.

FIG. 55 is a diagram illustrating a comparison between a value of an indicator in the current state and a result of predicting how the indicator will change according to the present embodiment.

FIG. 56 is a diagram for illustrating a pattern of how an indicator changes according to the present embodiment.

FIG. 57 shows a relationship between a transition of an indicator of an individual and an action of the individual for improvement according to the present embodiment.

FIG. 58 shows a relationship between a transition of an indicator of a group of users who resemble the user of interest that have continued an action for improvement and the action for improvement according to the present embodiment.

FIG. 59 shows a transition of an indicator of a group of users who resemble the user of interest that have stopped an action for improvement according to the present embodiment.

FIG. 60 shows a plurality of patterns of how an indicator transitions in the future according to the present embodiment.

FIG. 61 shows an example of a display screen displayed at a display unit of an information communication terminal in an objective maintenance process according to the present embodiment.

### DESCRIPTION OF THE PREFERRED EMBODIMENTS

Hereinafter, an embodiment of a system for management by objectives will be described with reference to the drawings. In the following description, identical parts and components are identically denoted. Their names and functions are also identical. Accordingly, they will not be described redundantly.

[Concept]

“Improving a habit” means to change one's life which has been considered “normal” to a desirable state, and then make the one recognize that the desirable life as “normal” henceforward. A procedure to improve a habit is as follows: (1) Understand what state a desirable normal state “henceforward” is. (2) Understand what change is necessary from the normal state “up to now.” (3) Make small changes without fail without burden on yourself. (4) Recognize the normal state “henceforward” that has been arrived at as a “normal” state henceforward for yourself.

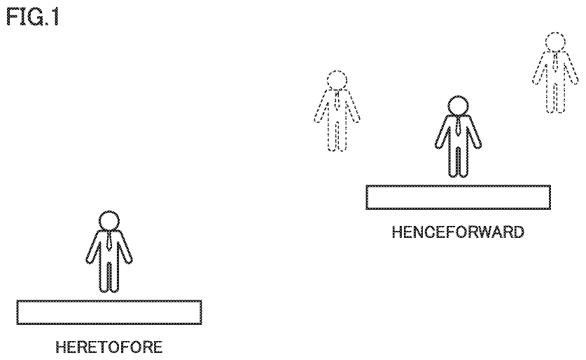

FIGS. 1 to 9 show steps 1 to 9, respectively, for improving a habit. Referring to FIG. 1, in step 1, a value is converted into a numerical value. In other words, once an objective is determined, a numerical objective for a prescribed indicator necessary for achieving the objective is specified.

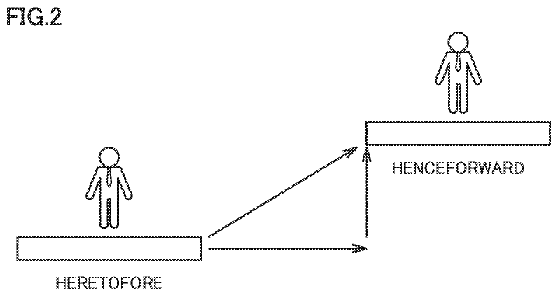

Referring to FIG. 2, in step 2, an amount of change up to the value is calculated. That is, the amount of change is calculated by setting a difference between the numerical objective and the current state, and a period of time until the achievement.

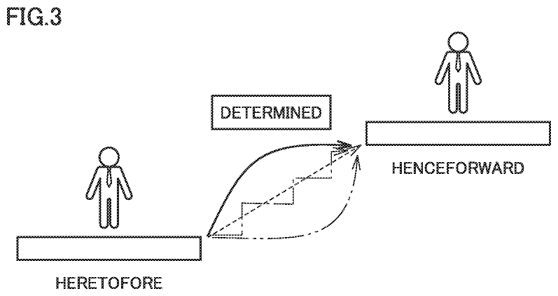

Referring to FIG. 3, in step 3, a route for realizing the change is presented. In other words, a plurality of routes are presented for the same amount of change, and which route to take is determined. A route represents a transition of a value of a prescribed indicator to a numerical objective of the prescribed indicator.

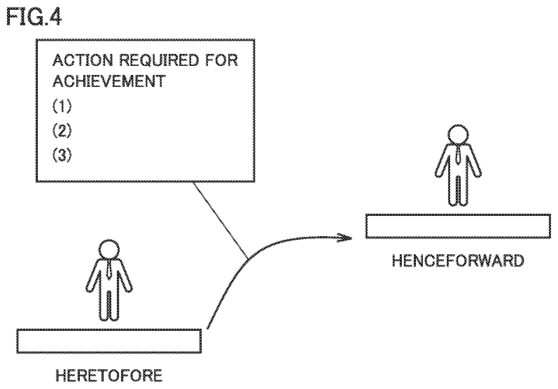

Referring to FIG. 4, in step 4, a specific action to proceed along the route is presented. That is, a specific action required to follow an assumed route is presented.

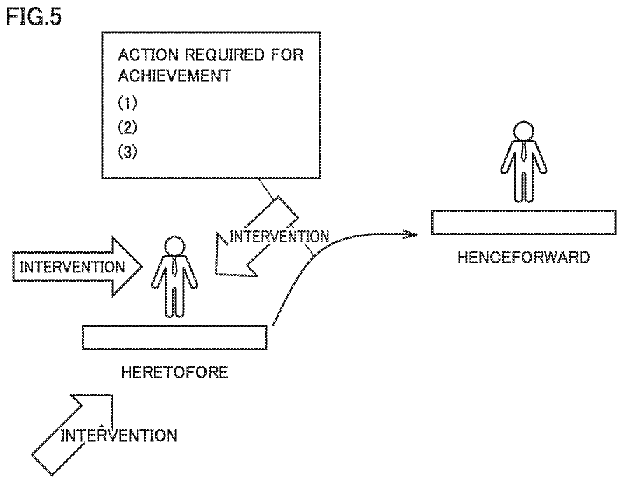

Referring to FIG. 5, in step 5, appropriate intervention is provided in an appropriate manner. In other words, intervention is performed with contents, at a time, at a place, and along a route, as selected as appropriate, to ensure that an action is performed.

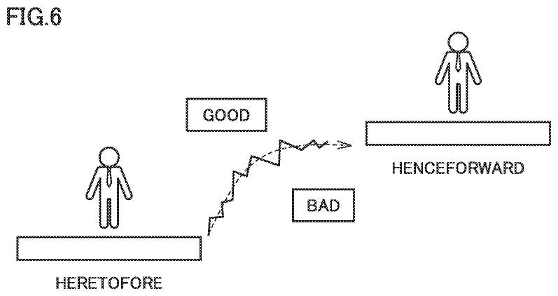

Referring to FIG. 6, in step 6, daily progress is confirmed and fed back. That is, intervention and daily progress thereby are evaluated against the determined route, and the contents of the intervention and the speed of change are changed depending on the progress.

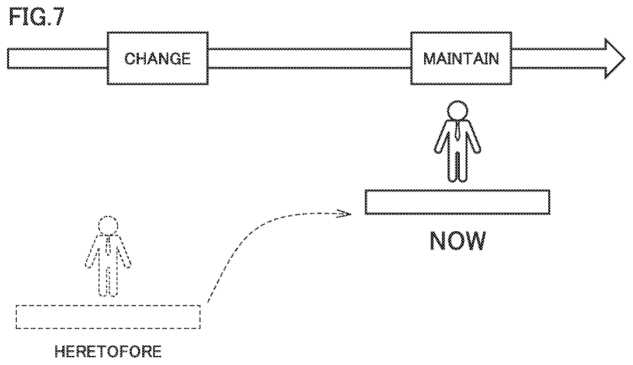

Referring to FIG. 7, in step 7, whether a stage is reached is determined and an intervention strategy is changed. In other words, when the objective is achieved, a phase is switched, and the approach is changed for “maintenance.”

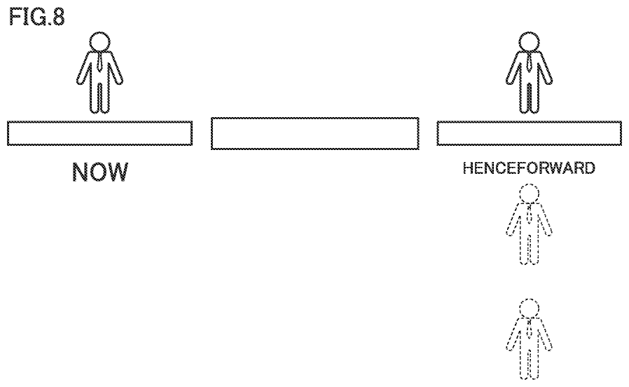

Referring to FIG. 8, in step 8, a descent future prediction and a route selection are performed. That is, maintenance of the current state or a gradual change is selected from a future in which a change is expected depending on an action in the future.

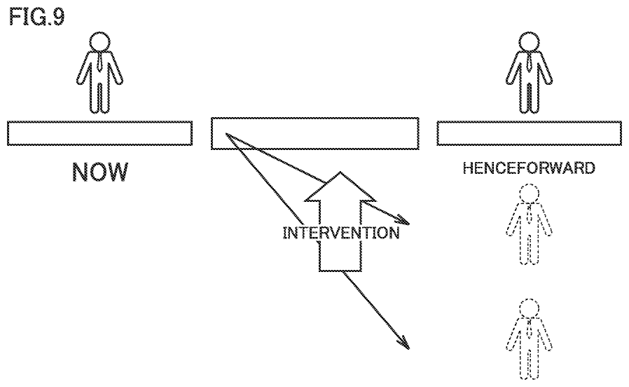

Referring to FIG. 9, in step 9, intervention is performed for slowing down a descent speed. In other words, a change that can deviate from a state of maintenance is assumed in advance, and intervention to avoid the change and praise for the maintenance are provided.

[System for Management by Objectives]

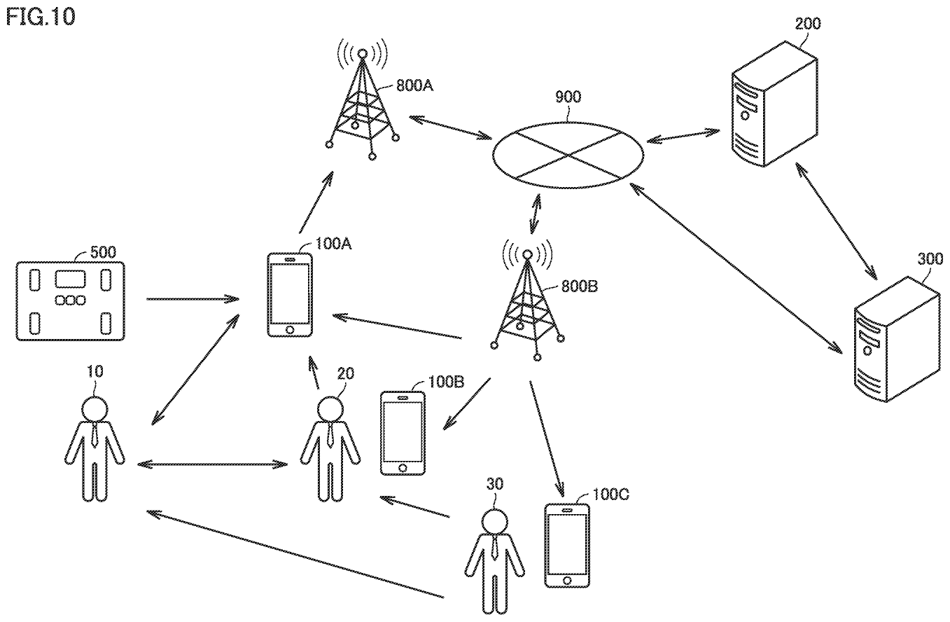

FIG. 10 shows a general configuration of a system for management by objectives according to the present embodiment. Referring to FIG. 10, the system for management by objectives includes information communication terminals 100A to 100C (for example, a smartphone, a mobile phone, a PC (a personal computer), a tablet PC, and the like) owned by users 10, 20, and 30, respectively, a server 200 for management by objectives, another server 300, a biological information measuring device 500, and communication facilities 800A and 800B of a telecommunications carrier which provides communications between the information communication terminals.

Servers 200 and 300 and communication facilities 800A and 800B are communicably connected to one another via a communication network 900 such as a public network such as the Internet and a public communication network, and a private network such as a LAN (Local Area Network). Information communication terminals 100A and 100B and communication facilities 800A and 800B are communicably connected to one another via wireless communication.

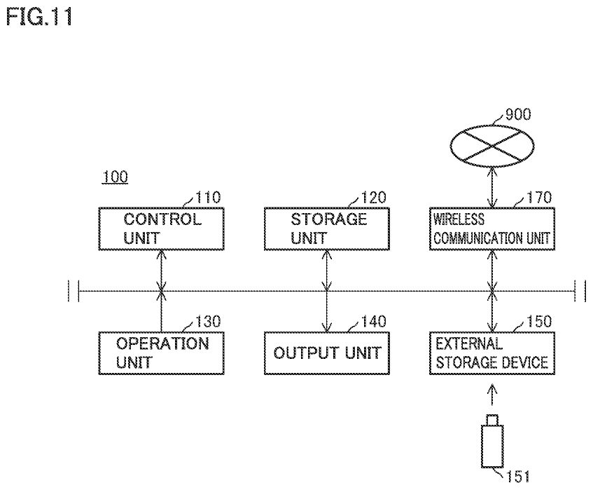

FIG. 11 is a block diagram showing a configuration of information communication terminal 100 according to the present embodiment. Referring to FIG. 11, information communication terminal 100 includes a control unit 110 for generally controlling information communication terminal 100, a storage unit 120 for storing prescribed information, an operation unit 130, an output unit 140, an external storage device 150, and a wireless communication unit 170. Although not shown, information communication terminal 100 also includes other components such as an audio input/output unit for inputting and outputting audio.

Control unit 110 includes a CPU (a central processing unit) and an auxiliary circuit therefor, and controls storage unit 120, operation unit 130, output unit 140 and wireless communication unit 170 to perform a prescribed process according to a program or data stored in storage unit 120, process data input from operation unit 130 and wireless communication unit 170, and store the processed data in storage unit 120 and output the processed data to output unit 140 and wireless communication unit 170.

Storage unit 120 includes a RAM (Random Access Memory) used as a work area necessary for control unit 110 to execute a program, and a ROM (Read Only Memory) for storing a program to be executed by control unit 110. Further, a program and data for performing a prescribed process are read from operation unit 130, wireless communication unit 160, or external storage device 150 and stored in the RAM. Further, a hard disk drive or a memory card may be used as an auxiliary storage device for assisting the storage area of the RAM.

External storage device 150 is configured by a memory card reader/writer. External storage device 150 electrically records prescribed data or a prescribed program received from control unit 110 in a storage medium 151 such as a memory card or a USB (Universal Serial Bus) memory, and reads the data or the program from storage medium 151 and passes it to control unit 110. External storage device 150 may be configured by a storage device such as a hard disk drive, a flexible disk drive, an MO (Magneto-Optical disk) drive, a CD (Compact Disc) drive, or a DVD (Digital Versatile Disk) drive.

Operation unit 130 includes a touch panel and operation buttons for inputting numbers, alphabets and other characters, such as telephone numbers and various data. Operation unit 130 may include a part for another operation. When operation unit 130 is operated by a user, an operation signal corresponding to an operation is transmitted from operation unit 130 to control unit 110. Control unit 110 controls each unit of information communication terminal 100 in response to the operation signal received from operation unit 130.

Wireless communication unit 170 is controlled by control unit 110 to receive a wireless signal from another information communication terminal 100 or a fixed telephone of the other party of a call via communication facility 800 of the telecommunications carrier and an antenna, convert the received wireless signal to an audio signal and transmit the audio signal to the audio input/output unit, and to receive an audio signal from the audio input/output unit and convert the audio signal to a wireless signal, and transmit the wireless signal to another information communication terminal 100 or the fixed telephone of the other party of the call via the antenna and communication facility 800 of the telecommunications carrier.

In addition, wireless communication unit 170 is controlled by control unit 110 to communicate with a device capable of data communication, e.g., a server or another information communication terminal 100, to receive a wireless signal via communication facility 800 of the telecommunications carrier and the antenna and convert the received wireless signal into data, and store the data in storage unit 120 and transmit the data to output unit 140 to display the data, and to convert data to be transmitted into a wireless signal and transmit the wireless signal to a server of a destination of data or another information communication terminal 100 via the antenna and communication facility 800 of the telecommunications carrier.

Wireless communication unit 170 is controlled by control unit 110 to communicate data with another network-communicable device, such as a server and another information communication terminal 100, via a public wireless LAN or a wireless LAN of a private network.

Output unit 140 includes a display and a speaker. Output unit 140 is controlled by control unit 110 so that information received by wireless communication unit 170, stored in storage unit 120 or read from storage medium 151 by external storage device 150, and converted into video and audio signals by control unit 110 is displayed by the display visibly and output by the speaker audibly, respectively.

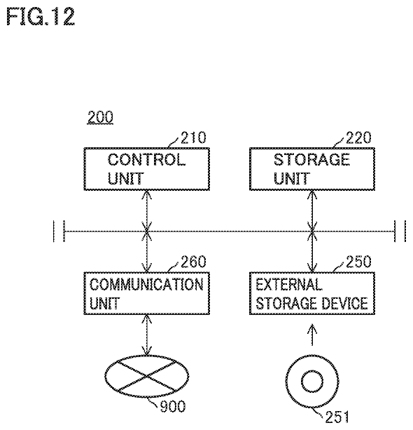

FIG. 12 is a block diagram showing a configuration of server 200 for management by objectives according to the present embodiment. Referring to FIG. 12, server 200 has a control unit 210 for generally controlling server 200, a storage unit 220 for storing prescribed information, an external storage device 250 assisting storage unit 220 for storing prescribed information, and a communication unit 260 for communicating with an external device via communication network 900.

Storage unit 220 is the same as storage unit 120 of information communication terminal 100 described with reference to FIG. 11, and thus will not be described repeatedly.

Communication unit 260 transmits and receives data to and from an external device via communication network 900 according to a predetermined protocol. Communication unit 260 externally transmits data received from control unit 210 and passes externally received data to control unit 210.

External storage device 250 is configured by a storage device such as a hard disk drive, a flexible disk drive, an MO drive, a CD drive, a DVD drive, or a memory card reader/writer. External storage device 250 magnetically, optically, or electrically records on storage medium 251 prescribed data or a prescribed program received from control unit 210 and reads the data or the program from storage medium 251 and passes it to control unit 210.

Storage medium 251 includes a hard disk, a flexible disk or a similar magnetic disk, CD-ROM (Compact Disk Read Only Memory), CD-R (Compact Disk Recordable), CD-RW (Compact Disk ReWritable), DVD-ROM (Digital Versatile Disk Read Only Memory), DVD-R (Digital Versatile Disk Recordable), DVD-RW (Digital Versatile Disk Rerecordable Disc), DVD-RAM (Digital Versatile Disk Random Access Memory), DVD+R, DVD+RW (Digital Versatile Disk ReWritable), BD-R (Blu-ray (registered trademark) Disc Recordable), BD-RE (Blu-ray (registered trademark) Disc Rewritable), BD-ROM (Blu-ray (registered trademark) Disc Read Only Memory) or a similar optical disk, MO or similar magneto-optical disk, a memory card, or USB memory Etc.

Control unit 210 has the same configuration as control unit 110 of information communication terminal 100 described with reference to FIG. 11. Control unit 210 controls storage unit 220, external storage device 250, and communication unit 260 to perform a prescribed process in accordance with a program and data stored in storage unit 220, and process data received from external storage device 250 or communication unit 260, store the processed data in storage unit 220 or storage medium 251 of external storage device 250 and/or output the data from communication unit 260.

While in the present embodiment server 200 includes neither an operation unit nor a display unit, and is operated by an operation through an operation unit of an external device to output information to a display unit of the external device, this is not exclusive and the sever may include a configuration of the operation unit and the display unit. The operation unit includes a keyboard and a mouse, and an operation signal indicating contents of an operation input to server 200 by operating the keyboard and the mouse of the operation unit may be passed to control unit 210. The display unit may include a display, and the display may display an image corresponding to image data received from control unit 210.

Note that another server 300 has a configuration similar to that of server 200, and accordingly, will not be described repeatedly.

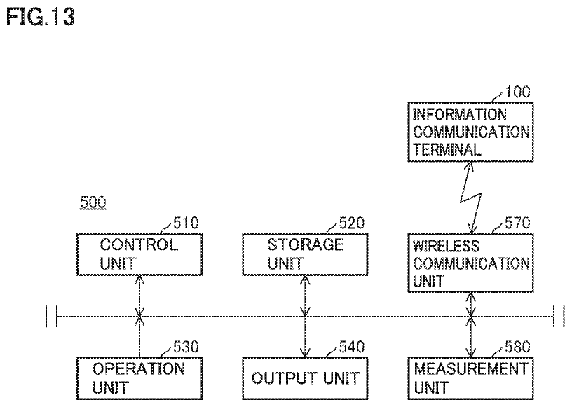

FIG. 13 is a block diagram illustrating a configuration of a biological information measuring device 500 according to the present embodiment. Referring to FIG. 13, biological information measuring device 500 such as a body composition meter as shown in FIG. 13 includes a control unit 510 for generally controlling measuring device 500, a storage unit 520 for storing prescribed information, an operation unit 530, an output unit 540, a wireless communication unit 570, and a measurement unit 580.

Control unit 510, storage unit 520, operation unit 530, output unit 540, and wireless communication unit 570 are similar to control unit 110, storage unit 120, operation unit 130, output unit 140 and wireless communication unit 170 of information communication terminal 100 described with reference to FIG. 11, and therefore, will not be described repeatedly. Wireless communication unit 570 may be capable of directly communicating with information communication terminal 100, or may be capable of communicating via communication network 900 or communication facility 800 of a telecommunications carrier.

Measurement unit 580 is controlled by control unit 110 to measure prescribed biological information out of a plurality of pieces of biological information of a user and transmit information of the measurement result to control unit 110. The biological information includes information indicating a state of a living body and information indicating the body's activity and movement, and specifically includes any indicators for a living body, such as a body weight, a chest circumference, an abdominal circumference, a body height, and a body composition value (body fat percentage, visceral fat level, subcutaneous fat percentage, basal metabolism, skeletal muscle percentage, muscle percentage, BMI, body age, and other values indicating a body composition), an amount of activity, a step count, a blood pressure value, a heart (or pulse) rate, a body temperature, a respiratory rate, indicator values for blood (a blood sugar value, an amount of neutral fat, an amount of cholesterol, and the like), calorie consumption, diet, water intake, excretion, sweating, vital capacity, sleep, and the like.

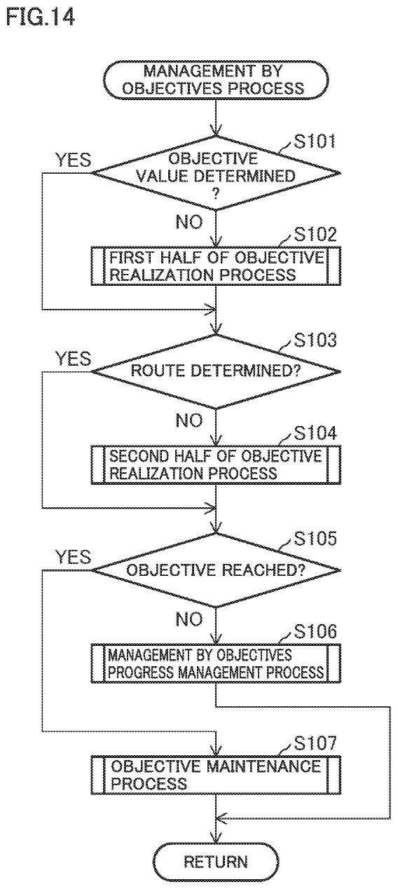

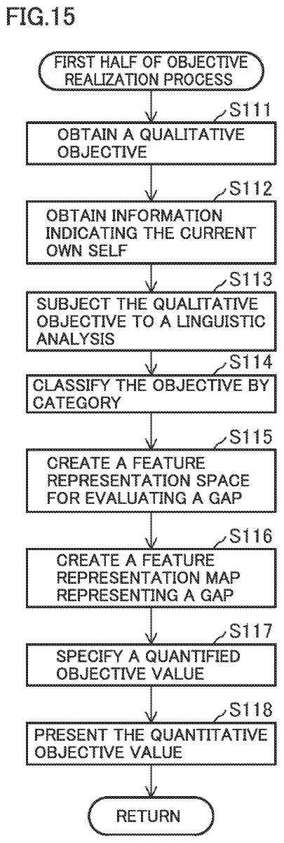

FIG. 14 is a flowchart of a management by objectives process performed by server 200 for management by objectives according to the present embodiment. Referring to FIG. 14, control unit 210 of server 200 determines whether an objective value of biological information for improvement has already been determined (step S101). When it is determined that the objective value has not been determined (NO in step S101), control unit 210 performs a first half of an objective realization process indicated in FIG. 15 described hereinafter (step S102).

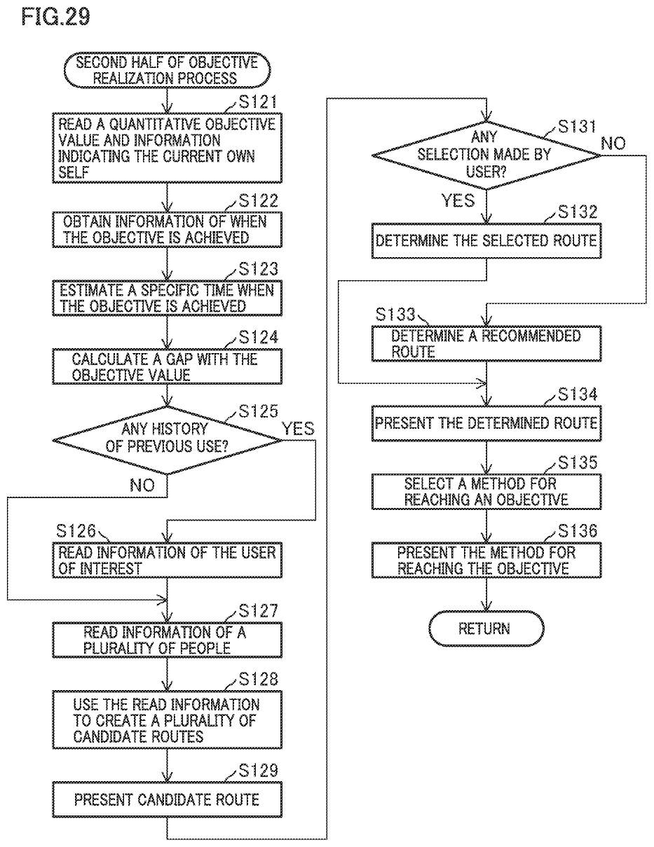

When it is determined that the objective value has been determined (YES in step S101), and after step S102, control unit 210 determines whether a route to reach the objective has already been determined (step S103). When it is determined that the route has not been determined (NO in step S103), control unit 210 performs a second half of the objective realization process indicated in FIG. 29 described hereinafter (step S104).

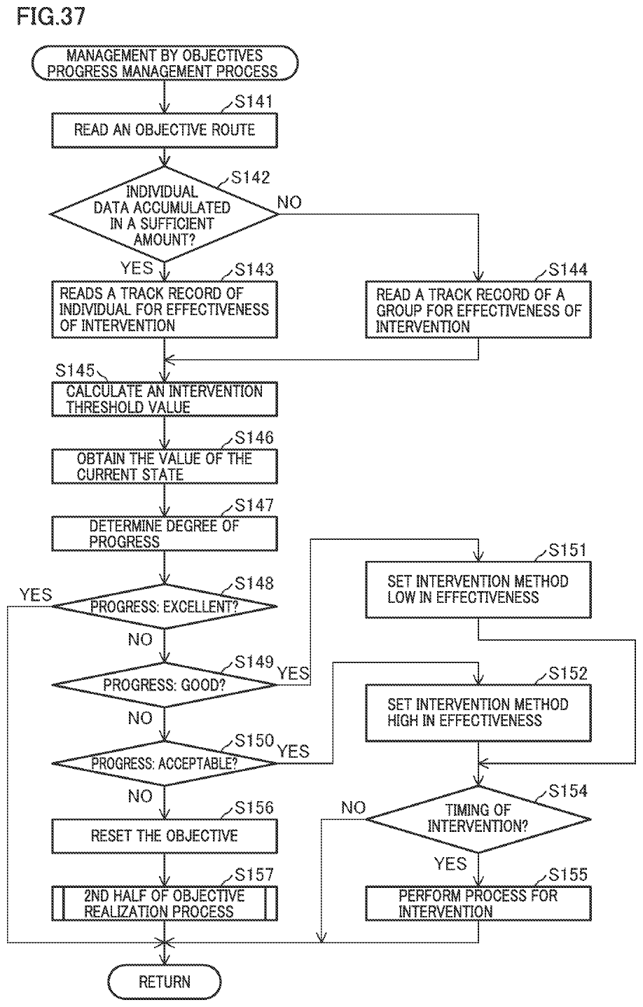

When it is determined that the route has been determined (YES in step S103), and after step S104, control unit 210 determines whether the determined objective has been reached (step S105). When it is determined that the objective has not been reached (NO in step S105), control unit 210 performs a management by objectives progress management process indicated in FIG. 37 described hereinafter (step S106).

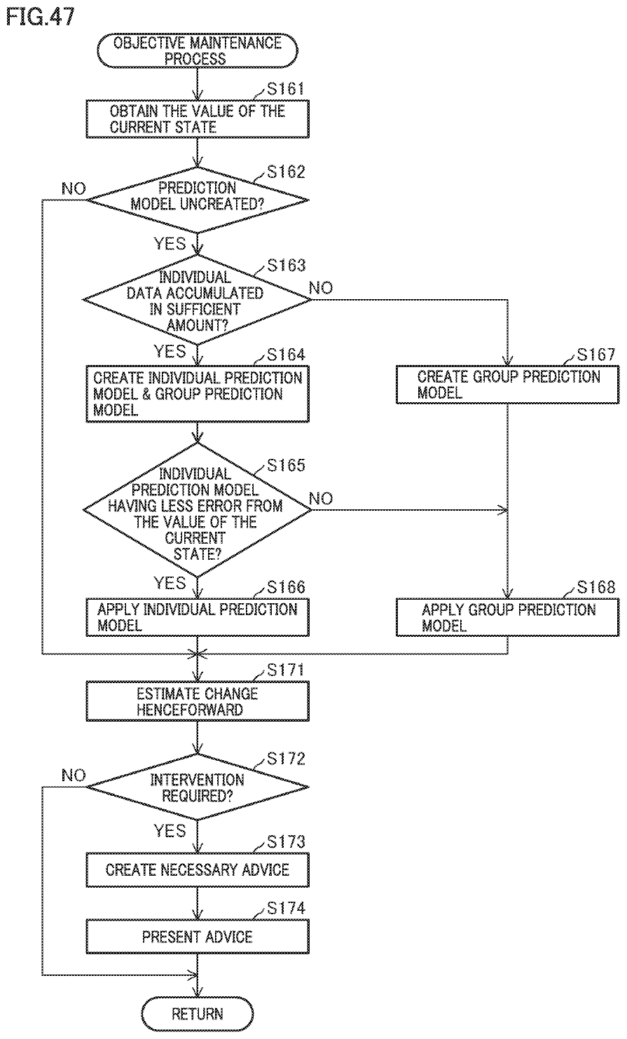

When it is determined that the objective has been achieved (YES in step S105), control unit 210 performs an objective maintenance process indicated in FIG. 47 described hereinafter (step S107).

[First Half of Objective Realization Process]

FIG. 15 is a flowchart of the first half of the objective realization process performed by server 200 for management by objectives according to the present embodiment. Referring to FIG. 15, control unit 210 of server 200 obtains a qualitative objective (step S111).

Specifically, control unit 210 obtains an intended qualitative objective that user 10 has input via information communication terminal 100A, and causes storage unit 220 to store such obtained information for each user. Such information may be input via information communication terminal 100A in any method, and for example, it may be input by hand, via voice, or interactively using hand or voice.

Further, control unit 210 obtains information indicating the current own self of the user (step S112). Specifically, control unit 210 obtains an attribute of the own self of user 10 (such as age, gender, family structure, and the like) input by user 10 via information communication terminal 100A, and causes storage unit 220 to store such obtained information for each user.

Subsequently, control unit 210 subjects to a linguistic analysis the qualitative objective obtained in step S111 (step S113). Specifically, the qualitative objective of user 10 input in characters is given a meaning through a morphological analysis or the like. Conventional techniques can be used for the morphological analysis.

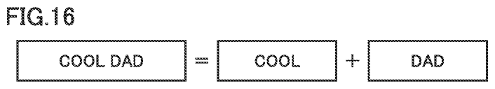

FIG. 16 is a diagram showing an example of the morphological analysis in the present embodiment. Referring to FIG. 16, in the morphological analysis, a language is divided into meaningful units. For example, when user 10 inputs “cool Dad” as a qualitative objective, it is divided into “cool” and “dad.”

Returning to FIG. 15, control unit 210 classifies the objective by category (step S114). More specifically, what the objective is like is classified by category (such as body composition, blood pressure, sleep, and the like) from an attribute of a meaning that the objective of the linguistic information analyzed in step S113 has.

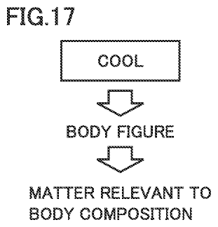

FIG. 17 is a diagram showing an example of classifying an objective by category in the present embodiment. Referring to FIG. 17, of the morphemes divided in step S113, the morpheme “cool” is a language relevant to a “body type,” and accordingly, it is classified into a category relevant to body composition.

Returning to FIG. 15, control unit 210 creates a feature representation space for evaluating a gap (step S115). More specifically, based on a category classified in step S114, a feature value for evaluating a gap with the objective is extracted and used to construct an axis for a multidimensional space. This multidimensional space is referred to as a feature representation space.

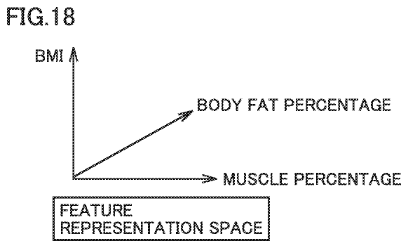

FIG. 18 is a diagram illustrating an example of the feature representation space according to the present embodiment. Referring to FIG. 18, for example, when storage unit 220 of server 200 for management by objectives has “BMI,” “body fat percentage” and “muscle percentage” stored therein as indicators relevant to a category for body composition, a feature representation space including an axis for “BMI,” an axis for “body fat percentage” and an axis for “muscle percentage” is created.

Returning to FIG. 15, control unit 210 creates a feature representation map representing a gap (step S116). More specifically, a range in the feature representation space created in step S114 is created as a feature representation map from the attribute of user 10 obtained in step S112 and the information of the meanings that the morphemes divided in step S113 have.

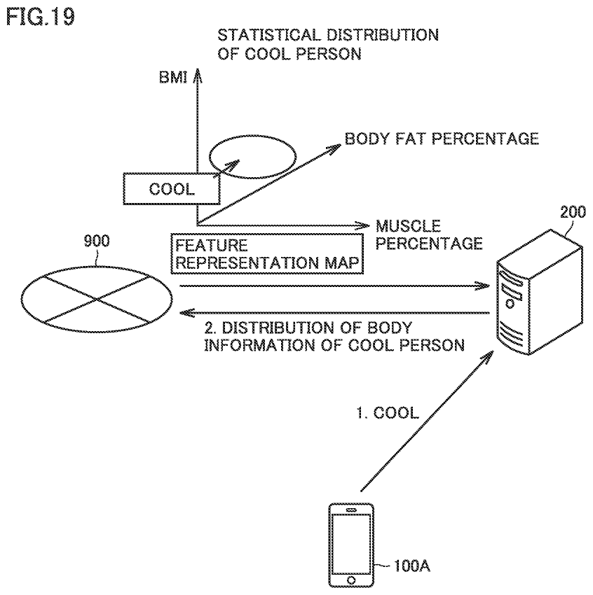

FIG. 19 is a first diagram illustrating an example of a process of creating a feature representation map according to the present embodiment. Referring to FIG. 19, server 200 previously obtains statistical information of a “cool” person from another server 300 or another information communication terminal 100 via communication network 900, and stores the information in storage unit 220 as a database of values for indicators for the “cool” person. Control unit 210 creates a distribution of the “cool” person in a feature representation space based on the statistical information of the “cool” person stored in storage unit 220.

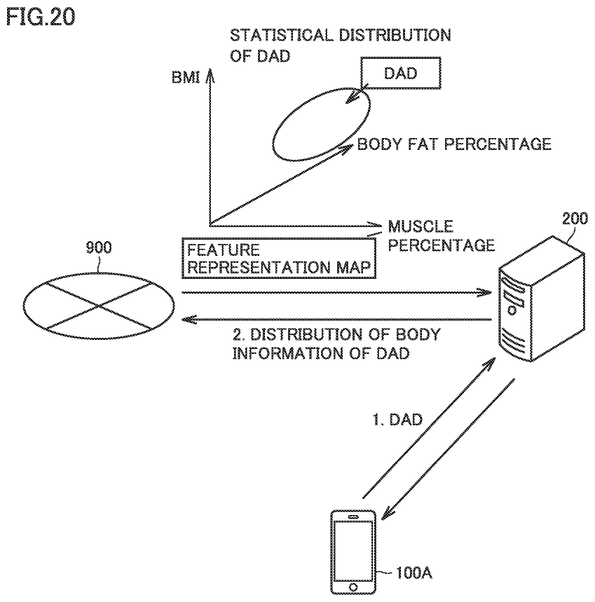

FIG. 20 is a second diagram illustrating an example of a process of creating a feature representation map according to the present embodiment. Referring to FIG. 20, server 200 previously obtains statistical information of “Dad” from another server 300 or another information communication terminal 100 via communication network 900, and stores the information in storage unit 220 as a database of values for indicators for “Dad.” Control unit 210 creates a distribution of “Dad” in a feature representation space based on the statistical information of “Dad” stored in storage unit 220.

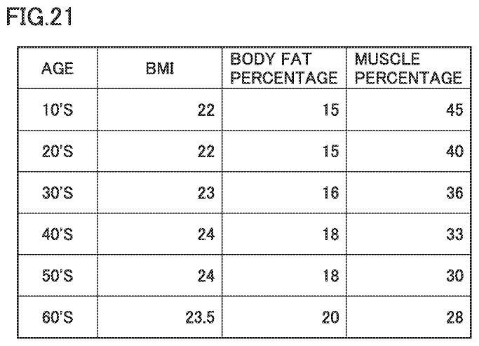

FIG. 21 shows an example of a database at an initial stage of a value of an indicator for a “cool” person's body composition according to the present embodiment. Referring to FIG. 21, in the initial stage of creating the database, server 200 stores values of indicators corresponding to a basic classification item (in this example, an item “age”) as a database as there is little data.

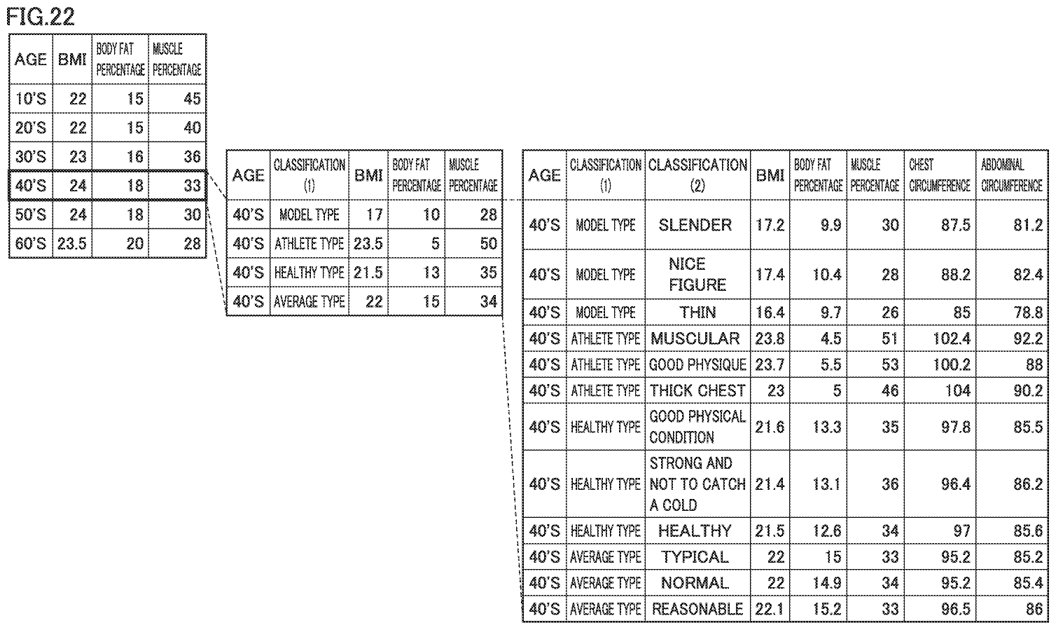

FIG. 22 shows an example of a database after data is accumulated for a value for an indicator for the “cool” person's body composition according to the present embodiment. Referring to FIG. 22, as data is accumulated and thus increased, server 200 can store values for indicators corresponding to another classification item (in this example, items “classification (1)” and “classification (2)”) as a database. In addition, it is possible to include values for a new indicator (in this example, chest circumference and abdominal circumference).

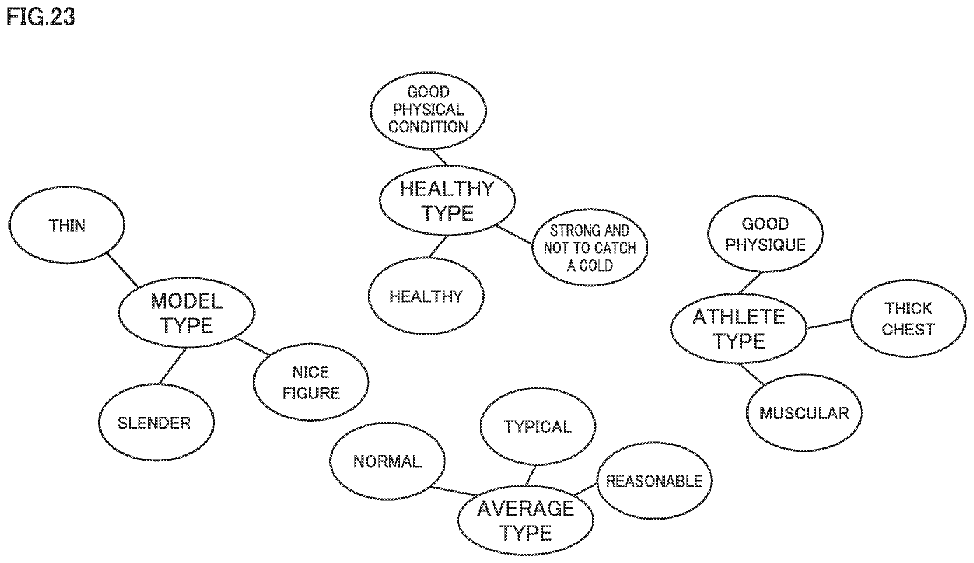

FIG. 23 shows an example of a language database for a category for a body composition according to the present embodiment. Referring to FIG. 23, if there is insufficient data in a database for indicators, then, as indicated in FIG. 22 by classification (1), abstracted classification items (model type, athlete type, healthy type, and average type) are adopted. For the abstraction, a language database organized by focusing on meanings that a language has, such as a thesaurus, as shown in FIG. 23, is used.

As the data in the database for indicators increases, then, as indicated in FIG. 22 by classification (2), more specific classification items (thin, nice figure, slender, good physique, thick chest, muscular, good physical condition, strong and not to catch a cold, healthy, normal, typical, reasonable) are used. Values for indicators for each category item are collected by referring to values of people who have achieved their objectives with the category items, synonyms and the like representing the objectives.

Returning to FIG. 15, control unit 210 specifies a quantified objective value (step S117). Specifically, the range of each feature value indicating the range of an overlapping portion of the ranges of the feature representation maps created in step S116 is specified as a quantitative objective value, and stored in storage unit 220.

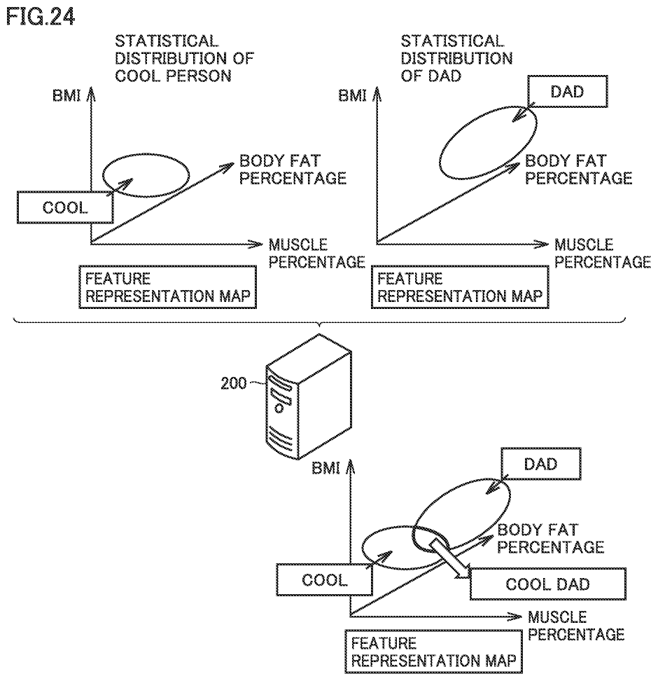

FIG. 24 is a first diagram illustrating an example of a process of specifying a quantitative objective value from feature representation maps according to the present embodiment. Referring to FIG. 24, when the feature representation map of a “cool” person shown in FIG. 19 and the feature representation map of “dad” shown in FIG. 20 are combined, a feature representation map of “cool dad” is obtained.

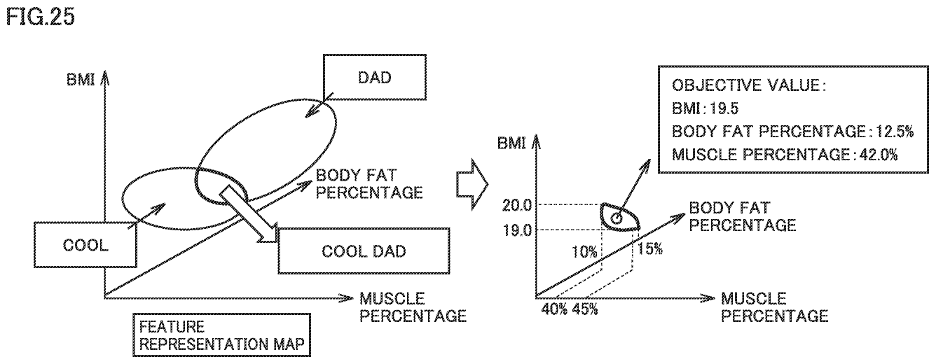

FIG. 25 is a second diagram illustrating an example of a process of specifying a quantitative objective value from a feature representation map according to the present embodiment. Referring to FIG. 25, the value of a point (in this example, a barycentric point) within the overlapping portion of the ranges of a plurality of (in this example, two) feature representation maps is set as a quantitative objective value.

While in this example a barycentric point is adopted as a quantitative objective value, it may be any other point within the range of the overlapping portion, and may be a point obtained by combining the median values of the ranges for the axes in the range of the overlapping portion (in FIG. 25, with a BMI of 19.0-20.0, a body fat percentage of 10-15 and a muscle percentage of 40-45, the objective values for BMI, body fat percentage, and muscle percentage are their respective median values, i.e., 19.5, 12.5 and 42.5, respectively.

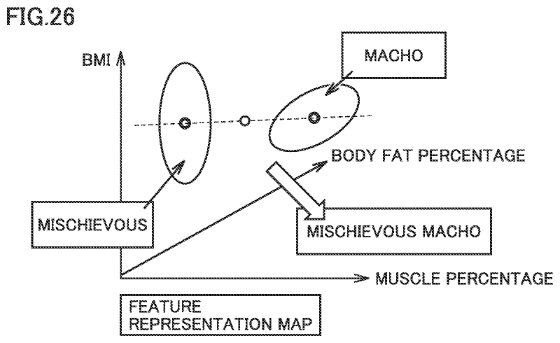

FIG. 26 is a third diagram illustrating an example of a process of specifying a quantitative objective value from a feature representation map according to the present embodiment. Referring to FIG. 26, when a plurality of feature representation maps do not have their respective ranges overlapping one another, the value of a point between the ranges (for example, the center point between the barycentric points of the ranges) is set as a quantitative objective value.

Returning to FIG. 15, control unit 210 transmits the quantitative objective value specified in step S117 to information communication terminal 100A to present the specified quantitative objective value at information communication terminal 100A of user 10 (step S118). Thereafter, control unit 210 returns a process to be performed to a process from which the current process is invoked.

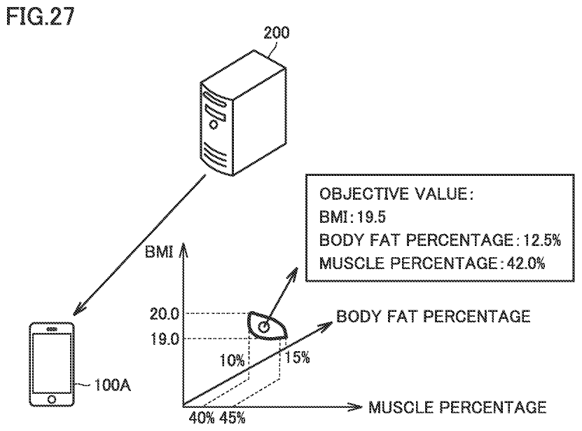

FIG. 27 is a diagram for illustrating presentation of a quantitative objective value according to the present embodiment. Referring to FIG. 27, a quantitative objective value is transmitted from server 200 for management by objectives to information communication terminal 100A of user 10.

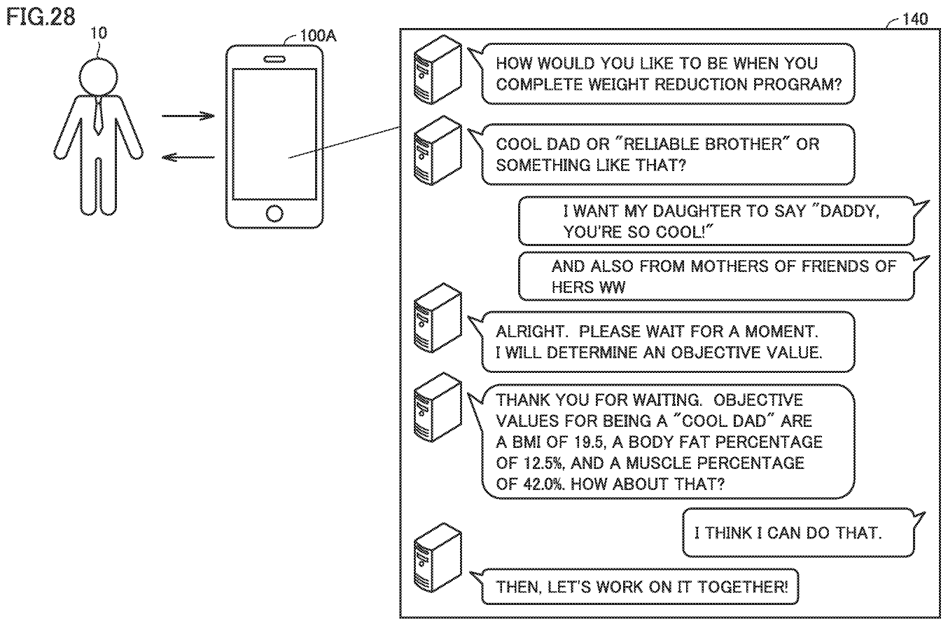

FIG. 28 shows an example of a display screen displayed at output unit 140 of information communication terminal 100A in the first half of the objective realization process according to the present embodiment. Referring to FIG. 28, the top balloon to the fifth balloon as counted from the top are displayed in step S111 of the first half of the objective realization process shown in FIG. 15. The sixth to eighth balloons as counted from the top are displayed in step S118 in FIG. 15.

[Second Half of Objective Realization Process]

FIG. 29 is a flowchart of a second half of the objective realization process performed by server 200 for management by objectives according to the present embodiment. Referring to FIG. 29, control unit 210 of server 200 reads a quantitative objective value and information indicating the current own self stored in storage unit 220 in the FIG. 15 process (step S121).

Subsequently, control unit 210 obtains information of when the objective is achieved (step S122), and estimates a specific time when the objective is achieved (step S123).

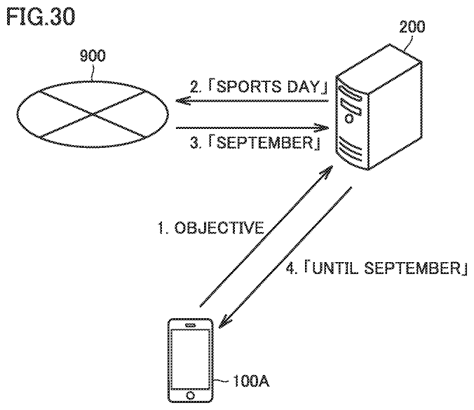

FIG. 30 shows an example of a process of obtaining information of when an objective is achieved according to the present embodiment. Referring to FIG. 30, for example, when user 10 inputs information indicating “I want to show my daughter at a sports day that I am athletic” as an objective to information communication terminal 100A, control unit 210 of server 200 determines that the daughter's “sports day” will be held in “September” based on information of another server 300 and the like connected to communication network 900. Thus, the deadline to achieve the objective is set to be “until September.”

In addition, when user 10 inputs information that “I want to wear a T-shirt neatly in July” as an objective, control unit 210 sets July as a time to achieve the objective.

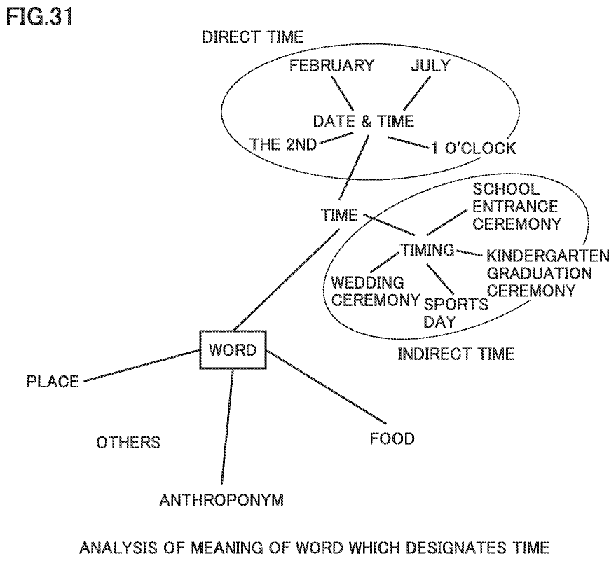

FIG. 31 is a diagram illustrating an example of a semantic analysis of a word designating a time according to the present embodiment. Referring to FIG. 31, such words include words for time in addition to words for places, words for food, words for personal names, and other words. Words for time include words for date and time as direct time, e.g., February, July, the second, 1 o'clock, and so forth. Words for time as indirect time include a school entrance ceremony, a kindergarten graduation ceremony, an athletic meet, a wedding ceremony, and so forth. Natural language processing is used to classify words for tense and subject when to achieve an objective to a cluster analysis to determine a time limit for achieving the objective.

Thus, control unit 210 specifies and obtains information for a time limit for achieving an objective from information input by user 10 via information communication terminal 100A, and causes storage unit 220 to store such obtained information for each user. When the information for the time limit for achieving the objective is a direct time limit, the information is used as it is, whereas when the information is an indirect time limit, a time limit is estimated.

Returning to FIG. 29, control unit 210 calculates a gap between the quantitative objective value stored in storage unit 220 and the information indicating the current own self (step S124).

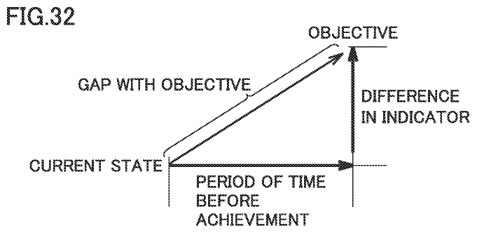

FIG. 32 is a diagram showing a gap with an objective value in the present embodiment. Referring to FIG. 32, the gap with the objective is indicated by the period before the objective is achieved and the difference that an indicator has between the current state and the objective. In FIG. 29 at step S124, for example, when the own self as of February has a body weight of 65 kg and a body fat percentage of 30%, and an objective is set to a body weight of 62 kg and a body fat percentage of 25% to be achieved by September, then, a period of 7 months, a body weight of −3 kg, and a body fat percentage of −5% are calculated as a gap.

Returning to FIG. 29, control unit 210 determines, based on information stored in storage unit 220 and the like, whether user 10 has a history of having previously used the system for management by objectives (Step S125). When it is determined that user 10 has previously used the system (YES in step S125), control unit 210 reads information of a process in which user 10 has succeeded in the past in reaching an objective or a process in which user 10 has failed in the past to do so (step S126).

When it is determined that user 10 has no such history (NO in step S125), and after step S126, control unit 210 reads information of a plurality of persons stored in storage unit 220 other than user 10 that is close to the condition of user 10 of this time (e.g., information with an approximate gap with an objective value, information with an objective approximate to that of user 10, and information with an attribute approximate to that of user 10) (step S127).

Then, control unit 210 uses the information read in steps S126 and S127 to create a plurality of candidate routes to reach the objective (step S128).

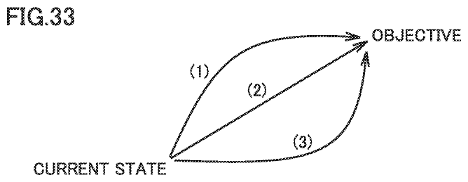

FIG. 33 is a diagram illustrating an example of a route to reach an objective according to the present embodiment. Referring to FIG. 33, as a candidate route to reach an objective, there are a plurality of patterns such as a straight type as indicated by a route (2), a curved type as indicated by routes (1) and (3), and a stepped type, and they each have a rate of change varying with a gap, a period, an individual's attribute and personality, and the like.

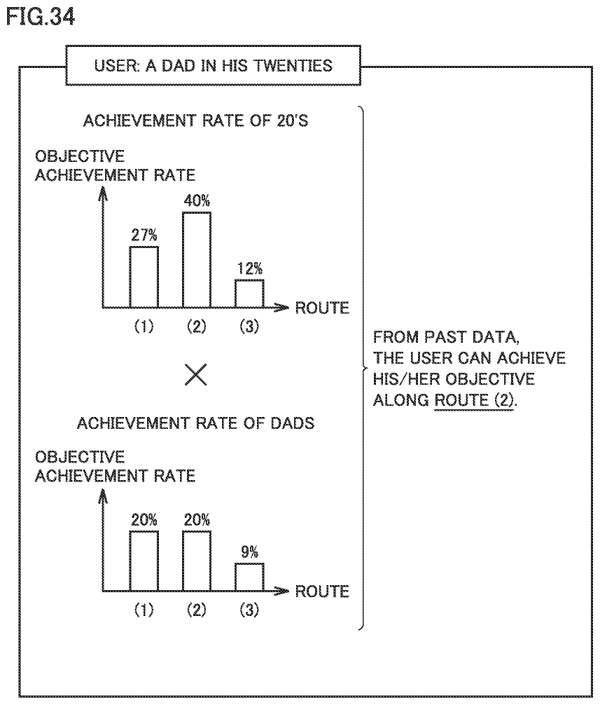

FIG. 34 is a diagram illustrating an example of a process of determining a recommended route to reach an objective according to the present embodiment. Referring to FIG. 34, a plurality of users' attributes, the plurality of users' objectives for their bodies, and information indicating a tendency of whether each user has reached his/her objective for the body are associated and stored in storage unit 220 of sever 200.

For example, when user 10 is a “dad in his twenties,” storage unit 220 stores that other users who set an objective identical or similar to that of user 10 and have an attribute identical to that of user 10, or are “in their twenties,” have objective achievement rates of 27%, 40% and 12% for routes (1), (2) and (3), respectively, and other users who set an objective identical or similar to that of user 10 and have an attribute identical to that of user 10, or are a “dad,” have objective achievement rates of 20%, 20% and 9% for routes (1), (2) and (3), respectively. Based on this, an average value of the objective achievement rates for each route is calculated and the route (2) having the highest average value is determined as a recommended route.

Returning to FIG. 29, control unit 210 transmits the candidate routes created in step S128 to information communication terminal 100A in order to present it on information communication terminal 100A of user 10 (step S129). Control unit 210 determines whether user 10 has selected a candidate route, that is, whether information indicating a selected candidate route has been received from information communication terminal 100A of user 10 (step S131).

When it is determined that a candidate route has been selected (YES in step S131), control unit 210 determines the selected candidate route as a route used for management by objectives (step S132). In contrast, when it is determined that no candidate route has been selected (NO in step S131), control unit 210 determines such a recommended route as shown in FIG. 34 as a route used for management by objectives (step S133).

Control unit 210 transmits an objective route determined in step S132 or step S133 to information communication terminal 100A of user 10 to present the objective route at information communication terminal 100A of user 10 (step S134).

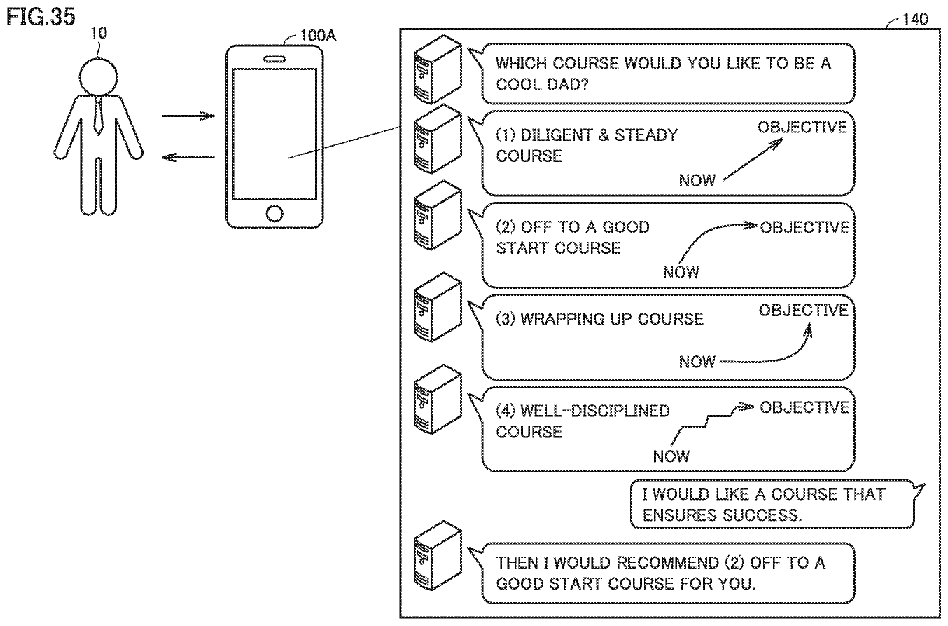

FIG. 35 shows an example of the display screen displayed at output unit 140 of information communication terminal 100A in the second half of the objective realization process according to the present embodiment. Referring to FIG. 35, the top balloon to the fifth balloon counted from the top balloon are displayed in the FIG. 29 step S129. The sixth and seventh balloons counted from the top balloon are displayed in the FIG. 29 step S134.

Returning to FIG. 29, control unit 210 selects a method for reaching an objective (step S135). More specifically, a method for reaching an objective is selected from a past history, user 10's attributes and preferences, and so forth. For example, for a plurality of users in their twenties, reducing food consumption is effective in improving a body composition (e.g., body weight). However, from information indicating that user 10 has many histories of walking as a method for reaching an objective according to a past history, running for 20 minutes every day is selected as a first recommendation and reducing food consumption is selected as a second recommendation.

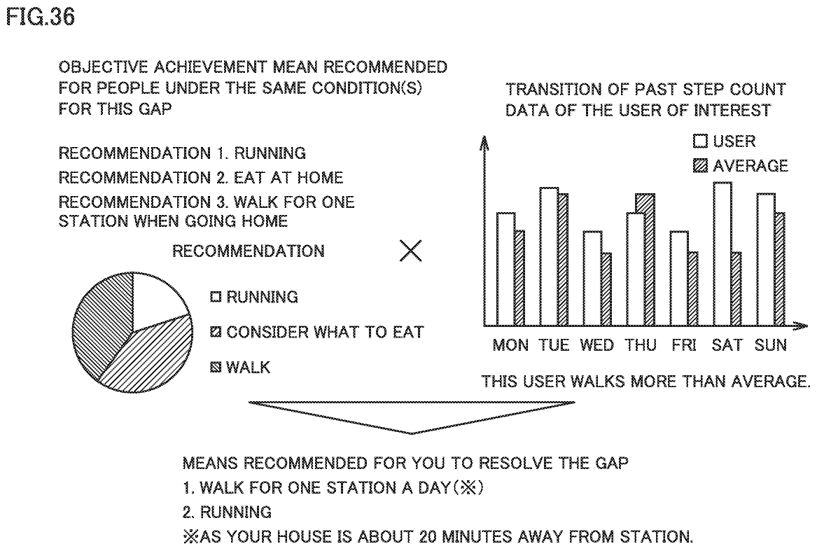

FIG. 36 shows an example of a process of selecting a method for reaching an objective according to the present embodiment. With reference to FIG. 36, a means for reaching an objective that is recommended for a person under a condition similar to a gap with an objective value that user 10 has is firstly running, secondly eating at home, and thirdly walking for one station when going home.

Further, from a transition of past step count data of user 10, it is determined that user 10 walks more than the average. From these facts, as a means for reaching an objective that is recommended for user 10 to resolve the gap with the objective value, walking for one station a day is firstly selected as user 10's house is about 20 minutes from the station, and running is secondly selected.

Returning to FIG. 29, control unit 210 transmits a method for reaching an objective that is determined in step S135 to information communication terminal 100A of user 10 in order to present the method at information communication terminal 100A of user 10 (step S136). Thereafter, control unit 210 returns a process to be performed to a process from which the current process is invoked.

[Management by Objectives Progress Management Process]

FIG. 37 is a flowchart of a management by objectives progress management process performed by server 200 for management by objectives in the present embodiment. Referring to FIG. 37, control unit 210 of server 200 reads an objective route stored in storage unit 220 in the FIG. 29 process (step S141).

Subsequently, control unit 210 determines whether individual data of a track record of intervention is accumulated in a sufficient amount (for example equal to or more than a number necessary for statistical calculation) (step S142). When it is determined that the data is sufficiently accumulated (YES in step S142), control unit 210 reads a track record of user 10 accumulated in storage unit 220 for effectiveness of intervention (step S143). When it is determined that the data is insufficiently accumulated (NO in step S142), control unit 210 reads a track record of a group accumulated in storage unit 220 for effectiveness of intervention (step S144).

Then, control unit 210 uses the read track record for effectiveness of intervention to calculate an intervention threshold value and an intervention method (step S145).

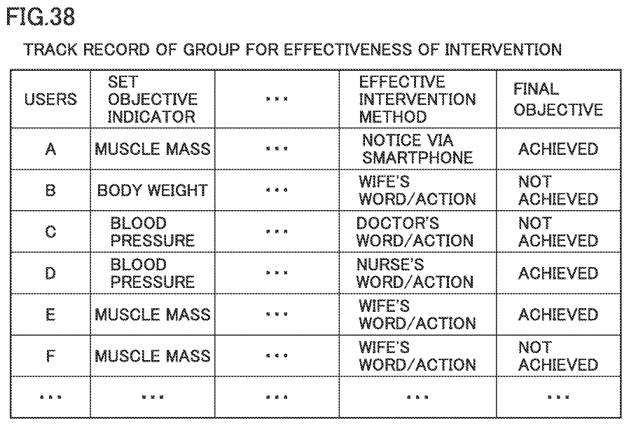

FIG. 38 shows an example of a track record of a group for effectiveness of intervention according to the present embodiment. Referring to FIG. 38, information indicating effectiveness of intervention, such as an effective intervention method, whether an objective has finally been achieved, and the like, is associated with a set objective indicator, gender, age, family structure and so forth for any user of the present system for management by objectives, and stored in storage unit 220.

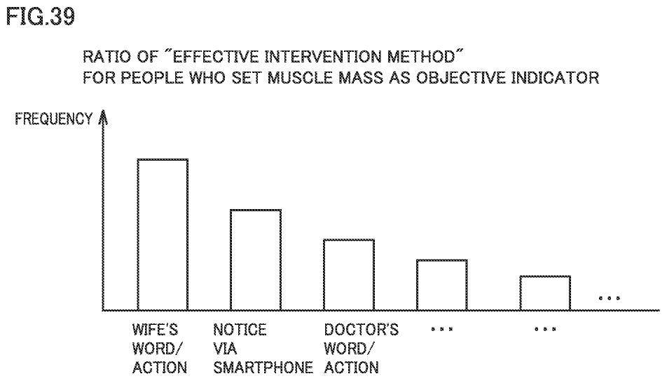

FIG. 39 is a diagram for illustrating a process of calculating a method for intervention by using a track record of a group according to the present embodiment. Referring to FIG. 39, from a track record of a group for effectiveness of intervention as shown in FIG. 38, any with “final objective” “achieved” is accumulated for each “set objective indicator.” In this example, a frequency of an “effective intervention method” for a person who has the same “set objective indicator” as user 10, or muscle mass, is highest for his wife's words and actions, followed by notification via a smart phone and a doctor's words and actions. Thus, as indicated by this example, an effective intervention method for user 10 is calculated from track records of users having an objective indicator identical to that of user 10.

In this example, as a method for intervention for user 10, a statistically effective intervention method is calculated from a method for intervention for a person having an objective indicator identical to that of user 10. However, this is not exclusive, and as a method for intervention for user 10, a statistically effective intervention method may be determined from a method for intervention for a person having an objective indicator approximate to that of user 10. The approximate objective indicator is stored in storage unit 220 in advance. For example, body fat percentage, visceral fat level, and subcutaneous fat percentage are stored as being approximate to each other.

Further, in the present embodiment, as a method for intervention for user 10, a statistically effective intervention method is determined from a method for intervention for a person having an objective indicator approximate in type to that of user 10. However, as an intervention method for user 10, a statistically effective intervention method may be determined from a method for intervention for a person having an objective indicator approximate in type and value to that of user 10. The objective indicator has an approximate value in a range, which is stored in storage unit 220 in advance. For example, storage unit 220 stores that an objective indicator that is a body weight has an approximate value in a range of ±10% of that body weight.

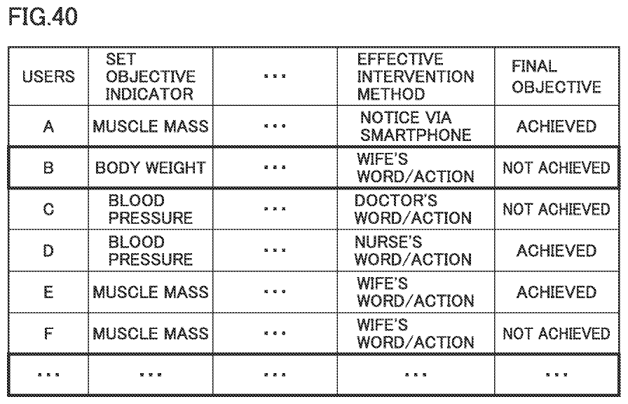

FIG. 40 is a first diagram for illustrating a process of calculating an intervention threshold value by using a track record of a group according to the present embodiment. Referring to FIG. 40, of a track record of a group for effectiveness of intervention as shown in FIG. 38, any user with “set objective indicator” (in this example, “body weight”) identical to that of user 10 is extracted. Then, the transition of the objective indicator of any extracted user that has achieved an objective is read from a database stored in storage unit 220 and a threshold value is statistically calculated.

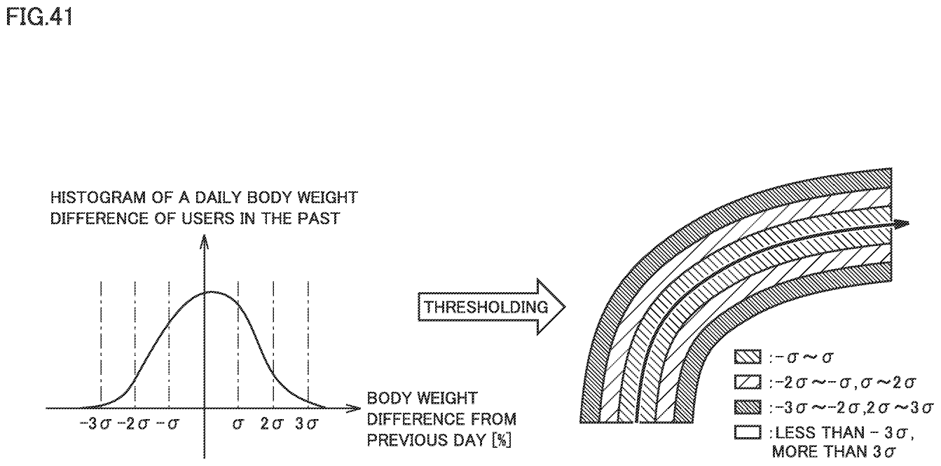

FIG. 41 is a second diagram for illustrating the process of calculating the intervention threshold value by using the track record of the group according to the present embodiment. Referring to FIG. 41, as a method for statistically calculating a threshold value, initially, a histogram of a daily difference of an objective indicator of a read plurality of users in body weight from the previous day is created. Subsequently, a standard deviation σ for the histogram is calculated. Then, −3σ, −2σ, −σ, +σ, +2σ, and +3σ are set as intervention threshold values for a value of an indicator along an objective route for user 10. In this way, an intervention threshold value for user 10 is calculated from a track record of users having an objective indicator identical to that of user 10.

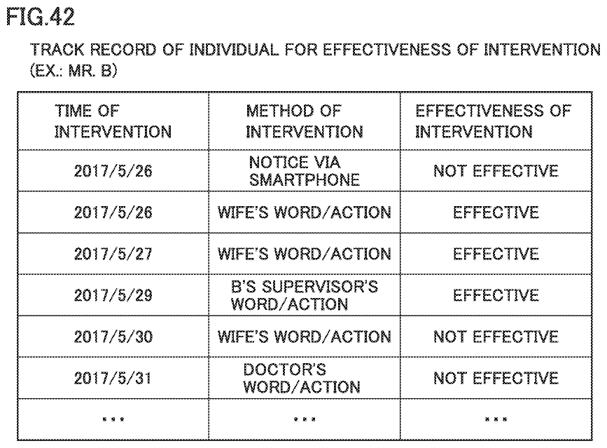

FIG. 42 shows an example of a track record of an individual for effectiveness of intervention according to the present embodiment. Referring to FIG. 42, information indicating effectiveness of intervention, such as a time of intervention, a method for intervention, and the presence or absence of an effect of intervention, are associated with each user of the system for management by objectives and thus stored in storage unit 220.

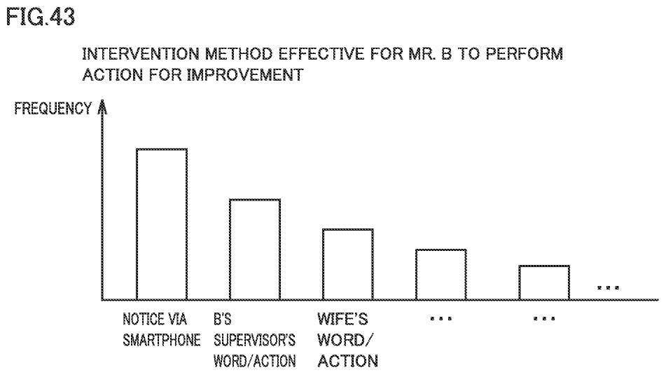

FIG. 43 is a diagram for illustrating a process of calculating a method for intervention by using a track record of an individual according to the present embodiment. Referring to FIG. 43, how frequently intervention has been effective is accumulated for each intervention method from a track record of an individual for effectiveness of intervention as shown in FIG. 42. In this example, a method for intervention that is most frequently effective in that Mr. B performs an action for improvement is notification via a smartphone, followed by his supervisor's words and actions and his wife's words and actions. Thus, as indicated by this example, an intervention method that is statistically effective for user 10 is calculated from a track record of user 10.

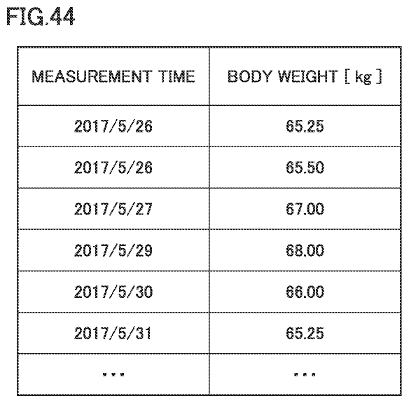

FIG. 44 is a first diagram for illustrating a process of calculating an intervention threshold value by using a track record of an individual according to the present embodiment. Referring to FIG. 44, when an objective indicator of user 10 is body weight, how the objective indicator of user 10 transitions is read from a database stored in storage unit 220, and a threshold value is statistically calculated therefrom.

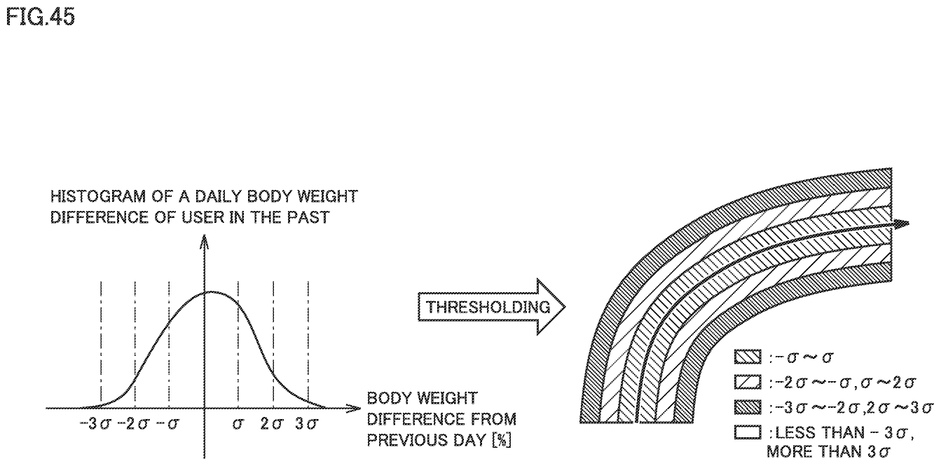

FIG. 45 is a second diagram for illustrating the process of calculating the intervention threshold value by using the track record of the individual according to the present embodiment. Referring to FIG. 45, as a method for statistically calculating a threshold value, initially, a histogram of a daily difference of the objective indicator of user 10 in body weight from the previous day is created. Subsequently, a standard deviation σ for the histogram is calculated. Then, −3σ, −2σ, −σ, +σ, +2σ, and +3σ are set as intervention threshold values for a value of an indicator along an objective route for user 10. In this way, an intervention threshold value for user 10 is calculated from a track record of user 10.

Returning to FIG. 37, control unit 210 obtains the value of the indicator of the current state of user 10 (step S146). More specifically, control unit 210 obtains from information communication terminal 100A a value of an indicator of the current state of user 10 that user 10 inputs via information communication terminal 100A and a value of an indicator of the current state of user 10 that is obtained from measuring device 500 via information communication terminal 100A.

Subsequently, control unit 210 compares the obtained value of each indicator of the current state of user 10 with the value of the indicator at the present time along the objective route for user 10 and determines a degree of divergence of whether the value of the indicator of the current state of user 10 falls within an “ideal range” of −σ to σ, an “allowable range” of −2σ to −σ or σ to 2σ, a “limit range” of −3σ to −2σ or 2σ to 3σ, or a “failure range” of less than −3σ or more than 3σ to determine a degree of progress of management by objectives for user 10 (step S147). Specifically, degrees of divergence falling within the “allowable range,” the “limit range,” and the “failure range,” respectively, are determined as “excellent,” “good” and “acceptable” degrees, respectively, of progress.

Note that a degree of divergence is not limited to such stepwise degrees of divergence as an “allowable range,” a “limit range” and a “failure range,” and it may be any other degree that indicates a divergence between the value of an indicator of the current state of user 10 and the value of the indicator at the present time along an objective route for user 10, and it may for example be the value of the difference between the value of the indicator of the current state of user 10 and the value of the indicator at the present time along the objective route for user 10 or may be a ratio of the value of the indicator of the current state of user 10 to the value of the indicator at the present time along the objective route for user 10.

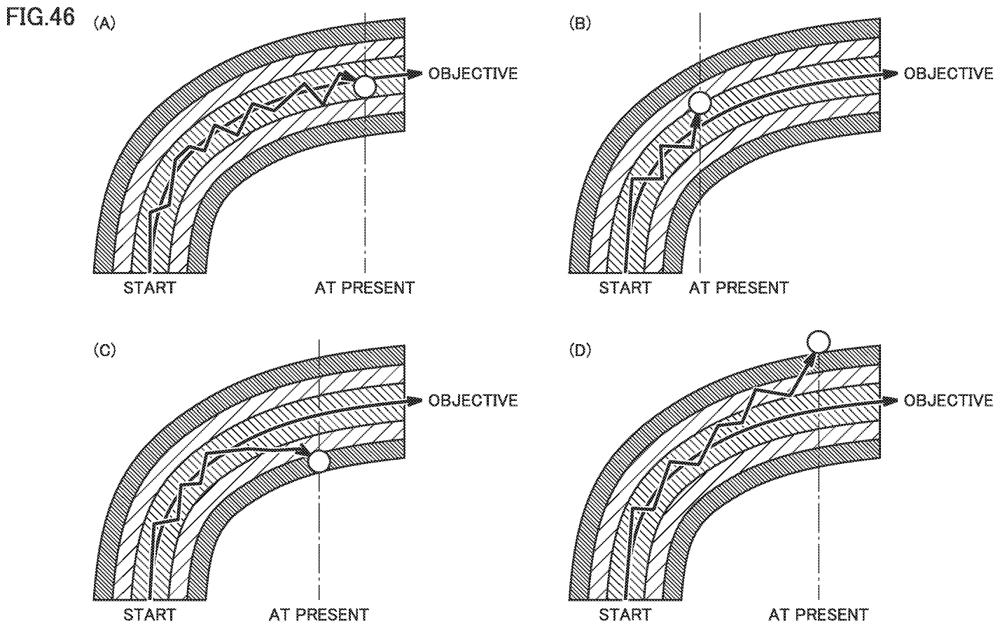

FIG. 46 is a diagram for illustrating a degree of progress of management by objectives according to the present embodiment. Referring to FIG. 46, FIGS. 46 (A) to 46 (D) show that a degree of progress of management by objectives by user 10, that is, the value of an indicator of the current state of user 10 falls within the “ideal range,” the “allowable range,” the “limit range” and the “failure range,” respectively.

Returning to FIG. 37, control unit 210 determines whether a degree of progress is “excellent,” that is, whether the value of the indicator of the current state of user 10 falls within the “ideal range” (step S148). When it is “excellent” (YES in step S148), control unit 210 returns a process to be performed to a process from which the present process is invoked.

When it is determined that the degree of progress is not “excellent” (NO in step S148), control unit 210 determines whether the degree of progress is “good,” that is, whether the value of the indicator of the current state of user 10 falls within the “allowable range” (step S149). When it is “good” (YES in step S149), control unit 210 sets an intervention method which is low in effectiveness (step S151).

When it is determined that the degree of progress is not “good” (NO in step S149), control unit 210 determines whether the degree of progress is “acceptable,” that is, whether the value of indicator of the current state of user 10 falls within the “limit range” (step S150). When it is determined to be “acceptable” (YES in step S150), control unit 210 sets an intervention method which is high in effectiveness (step S152).

After step S151 and step S152, control unit 210 determines whether a time to intervene has arrived (step S154). When it is determined that a time to intervene has arrived (YES in step S154), control unit 210 performs a process for intervention (step S155). After that, control unit 210 returns a process to be performed to a process from which the present process is invoked.

When an intervention method which is less effective is a typical message transmitted from server 200 to user 10 via information communication terminal 100A, an intervention method which is more effective is intervention with an atypical message issued from a person relevant to user 10 or an expert (e.g., a doctor, a trainer, and the like) in response to a request received from server 200.

When the intervention method which is less effective is a message issued from a person equivalent to or lower than user 10 in position (e.g., a family member (e.g., a spouse, a child, a parent, sibling), a friend or the like) in response to a request received from server 200, the intervention method which is more effective is intervention with a message issued from a person upper in position than user 10 (e.g., a supervisor at workplace, a senior, a teacher, and so forth) in response to a request received from server 200.

For each type of objective achievement method such as diet and exercise, an intervener having a high degree of contribution to user 10 and the objective achievement method is determined. A degree of contribution refers to a classification in grade for contribution in magnitude (e.g., large, medium, small). The intervener is determined by using environmental information such as user 10's family structure, work environment and the like, and user 10's or a plurality of other users' past practice data.

For example, a result of intervention by each intervener for user 10 for each type of objective indicator is previously stored in storage unit 220. Alternatively, a result of intervention by each intervener for each of a plurality of persons for each type of objective indicator is stored in storage unit 220. As a result of intervention is stored that intervention is successful when the indicator of the objective has an improved value after intervention and that intervention is unsuccessful when the indicator of the objective does not have an improved value after intervention. A result of intervention stored in storage unit 220 is used to determine a degree of contribution for an objective indicator in accordance with how many times intervention is successfully done to determine an intervener having a high degree of contribution.

A time to intervene may be a point in time predetermined for each type of objective achievement method, or information of timing for performing an objective achievement method may be obtained or estimated from user 10 via information communication terminal 100A to determine the time to intervene to be around timing for performing the objective achievement method depending on what is subject to intervention.

A result of intervention for an objective for each timing may be stored in storage unit 220 and used to determine a timing having a high contribution.

For example, a result of intervention for each timing of intervention for user 10 for each type of objective indicator is stored in storage unit 220. Alternatively, a result of intervention for each timing of intervention for each of a plurality of people for each type of objective indicator is stored in storage unit 220. As a result of intervention is stored that intervention is successful when the indicator of the objective has an improved value after intervention and that intervention is unsuccessful when the indicator of the objective does not have an improved value after intervention. A result of intervention stored in storage unit 220 is used to determine a degree of contribution for the objective indicator in accordance with how many times intervention is successfully done to determine a timing for intervention having a high degree of contribution.

When user 10 has a lifestyle varying between weekdays and holidays or the like, it is preferable that who performs intervention and when to intervene are determined according to how the lifestyle varies even for the same objective achievement method and content subject to intervention.

For example, when what is subject to intervention is “diet,” then, for a weekday which is a work day of user 10, immediately before timing of lunch, server 200 for management by objectives notifies a supervisor of user 10 at his/her workplace, that is, a person who is higher in position than user 10, to have the supervisor tell user 10 what menu user 10 should select for lunch to urge the supervisor to provide intervention for user 10 for “diet.”

When what is subject to intervention is “diet,” then, for a holiday which is a day off of user 10, before the spouse or wife of user 10 determines what she cooks for lunch, server 200 for management by objectives notifies the wife of a method for cooking lunch to urge the wife to provide intervention for user 10 for “diet.”

When what is subject to intervention is “exercise,” then, for a weekday which is a work day of user 10, before user 10 goes to workplace and back home, server 200 for management by objectives notifies information communication terminal 100A of user 10 of walking fast or using stairs or the like for exercise to thus provide intervention for user 10 for “exercise.”

When what is subject to intervention is “exercise,” then, for a holiday which is a day off of user 10, before the holiday, server 200 for management by objectives notifies a child of user 10 of information of exercise that user 10 can do on the holiday to urge the child to provide intervention for his/her father, or user 10, for “exercise.”

An intervention method having a high success rate for intervention for objective achievement methods of a plurality of people that are identical to the objective achievement method of user 10 (a telephone call from his wife when it has a highest success rate followed by a word from his daughter and notification via a smartphone) may be used for intervention. For example, server 200 for management by objectives urges his wife to perform intervention for “walking” serving as an objective achievement method to have his wife make a telephone call to user 10 and tell him a message “How would you like to walk from the station as the objective is nearing?”

Intervention may be performed in an intervention method that has been effective in intervention for a person having an attribute similar to that of user 10. For example, for a man in his forties for whom a word from his daughter is most effective, followed by informing his wife of designating a cooking method, and notifying him via a smartphone, the following may be considered.

Within a day in which a degree of progress is determined to be “acceptable,” server 200 for management by objectives may notify a daughter of user 10 of the current state of user 10 and ask her to say to user 10 “How are you doing these days?” to thus urge her to provide intervention for user 10. For intervention for diet, if the daughter has a conversation with her father for 18:00 to 19:00, then the daughter may be urged at 18:30 to provide intervention for user 10.

Within a day in which a degree of progress is determined to be “acceptable,” server 200 for management by objectives may notify user 10's wife of the current state of user 10 and also present an effective recipe to his wife to urge his wife to provide intervention for user 10's “diet.” If his wife considers a menu at 13:00, she may be urged at 12:30 to provide intervention for user 10's “diet.”

On or after a day after a day in which a degree of progress is determined to be “acceptable,” server 200 for management by objectives may make a contact with the user's smart phone for confirming a situation to provide intervention. If user 10 has meals at 6:00, 12:15, and 19:30, intervention may be done for user 10 at times immediately therebefore, that is, 5:45, 12:00, and 19:15, respectively.

Returning to FIG. 37, when a degree of progress is not determined to be “acceptable” (NO in step S150), control unit 210 resets the objective (step S156) and performs the second half of the objective realization process indicated in FIG. 29 described above (Step S157). When resetting the objective, the objective of the indicator indicated in FIG. 32 is changed depending on the degree of progress in the current state, or the deadline set for reaching the objective is extended. Thereafter, control unit 210 returns a process to be performed to a process from which the current process is invoked.

FIG. 47 is a flowchart of an objective maintenance process performed by server 200 for management by objectives according to the present embodiment. Referring to FIG. 47, control unit 210 of server 200 obtains the value of an indicator of the current state of user 10 (step S161). More specifically, control unit 210 obtains from information communication terminal 100A the value of an indicator of the current state of user 10 that user 10 inputs via information communication terminal 100A and the value of an indicator of the current state of user 10 that is obtained from measuring device 500 via information communication terminal 100A.

Subsequently, control unit 210 determines whether a prediction model has not been created (step S162). When it is determined that the prediction model has been created (NO in step S162), control unit 210 advances a process to be performed to step S171. When it is determined that the prediction model has not been created (YES in step S162), control unit 210 determines whether individual data of user 10 is accumulated in a sufficient amount (step S163).

When it is determined that the individual data is accumulated in a sufficient amount (YES in step S163), control unit 210 creates an individual prediction model and a group prediction model (step S164).

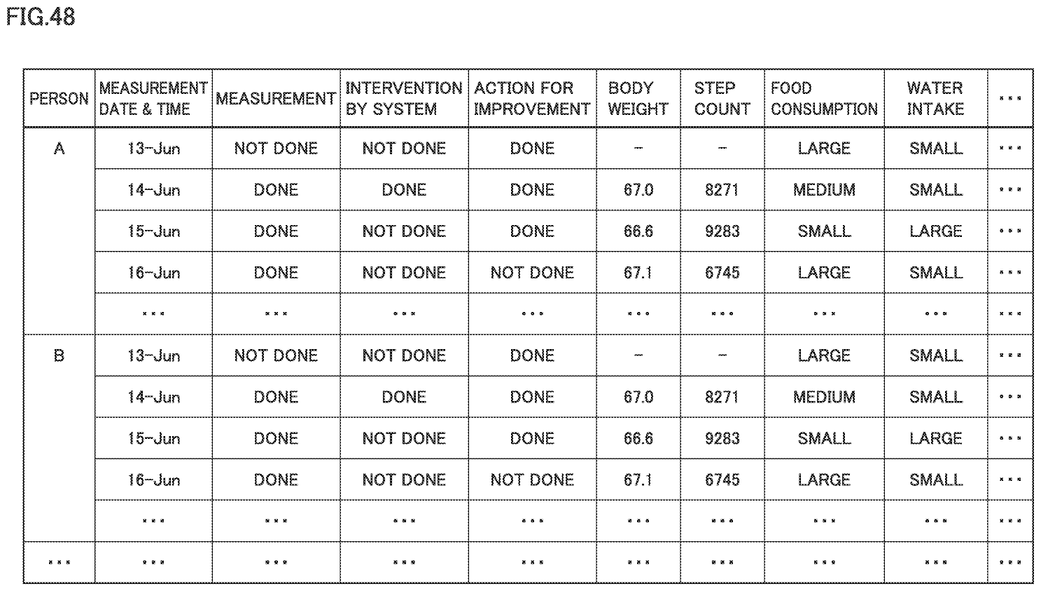

FIG. 48 shows past data of a group that is extracted as the data is similar to that of user 10 according to the present embodiment. With reference to FIG. 48, the data is extracted from past data of all users of the system for management by objectives, as the data is data of users similar to user 10 in that they set an objective similar to that of user 10 and have a tendency similar to that of user 10 in how an indicator (in this example, body weight) transitions.

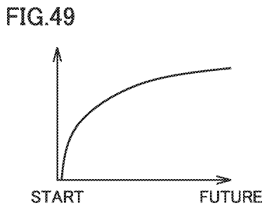

FIG. 49 represents a predicted transition of a change in an indicator of data similar to that of user 10 according to the present embodiment. Referring to FIG. 49, this graph is a graph showing an average transition and a confidence interval transition of an indicator (in this example, body weight) predicted from the data of FIG. 48. For example, it can be approximated by a relational expression (1) of y=a×x2+b×x+c.

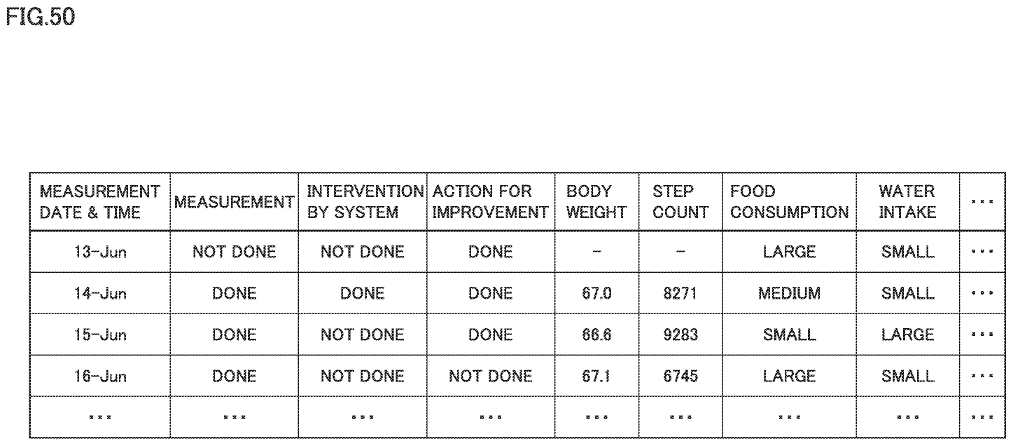

FIG. 50 shows an individual's past data extracted according to the present embodiment. Referring to FIG. 50, the data is data of user 10 extracted from past data of all users of the system for management by objectives.

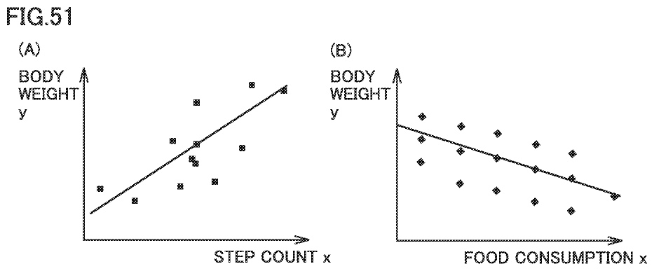

FIG. 51 is a diagram illustrating a predicted transition of a change in an indicator for user 10 according to the present embodiment. With reference to FIG. 51, the graph of FIG. 51(A) is a graph showing a correlation between user 10's step count and user 10's body weight based on the data of FIG. 50. The graph in FIG. 51(B) is a graph showing a correlation between user 10's food consumption and user 10's body weight based on the data of FIG. 50. From approximation lines in these graphs, a body weight prediction model represented by a relational expression (2) of y=a×step count+b×food consumption+ . . . +c can be obtained.

In this way, it is possible to calculate a degree of influence of each factor from past information of an individual and construct a prediction model. For example, for providing a prediction for one week ahead, a prediction model is constructed for each day. For providing a prediction for one month ahead, a prediction model is constructed for each week. For providing a prediction for three months or more ahead, a prediction model is constructed for each month.

Returning to FIG. 47, control unit 210 determines whether the individual prediction model has less error from the value of the current state than the group prediction model (step S165).

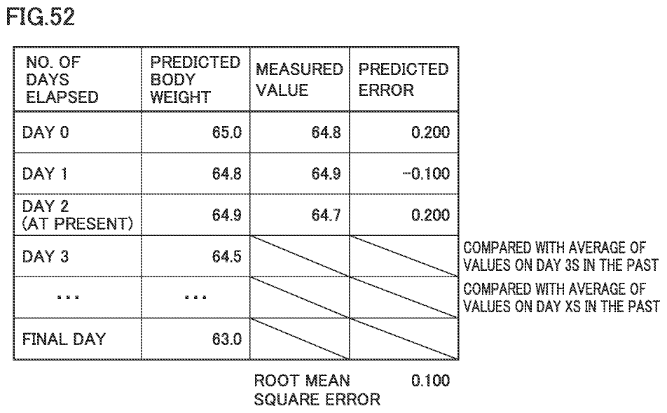

FIG. 52 shows an error evaluation using a group prediction model according to the present embodiment. Referring to FIG. 52, when it is day 2 now, then, for day 0 to day 2, a predicted error between a predicted body weight obtained by the above-described relational expression (1) and an actually measured value is calculated. For day 3 et. seq. each, a predicted error between an average of values on day 3s et. seq. in the past and a predicted body weight obtained from the relational expression (1) is calculated. A root mean square error of these predicted errors is calculated as an error when a group prediction model is used.

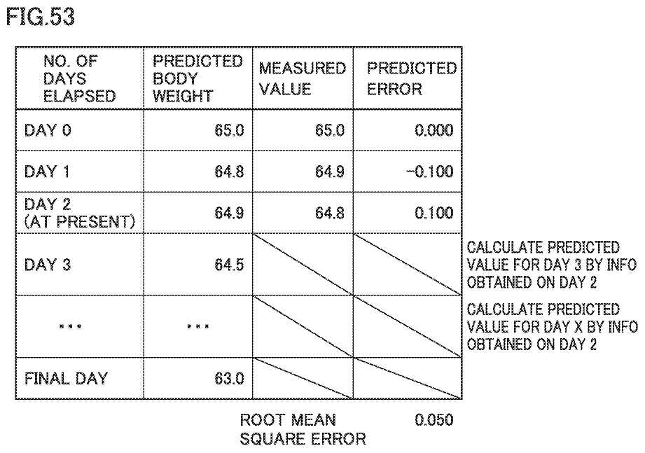

FIG. 53 shows an error evaluation using an individual prediction model according to the present embodiment. Referring to FIG. 53, when it is day 2 now, then, for day 0 to day 2, a predicted error between a predicted body weight obtained by the above-described relational expression (2) and an actually measured value is calculated. For day 3 et. seq., a value predicted for each day from information obtained on day 2 is calculated, and a predicted error between the calculated predicted value and a predicted body weight obtained by the above-described relational expression (2) is calculated. A root mean square error of these predicted errors is calculated as an error when the individual prediction model is used.

Returning to FIG. 47, in the case of the examples of FIGS. 52 and 53, it is determined in step S165 that the individual prediction model has less error. When it is determined that the individual prediction model has less error (YES in step S165), control unit 210 applies the individual prediction model as a model used for prediction (step S166). When it is determined that the group prediction model has less error (NO in step S165), control unit 210 applies the group prediction model as a model used for prediction (step S168).

When it is determined that the individual data does not have a sufficient amount (NO in step S163), control unit 210 creates a group prediction model (step S167), and applies the group prediction model as a model used for prediction (step S168).

Subsequently, control unit 210 estimates how the indicator of user 10 will change in the future (step S171). Specifically, from epidemiological information of the prediction model described above, how the indicator of user 10 will transition is simulated based on a transition made when a person similar to user 10 does not particularly change his/her activities and behaves in the same manner as before. In this simulation, an advance notice model applied in step S166 or step S168 is used.

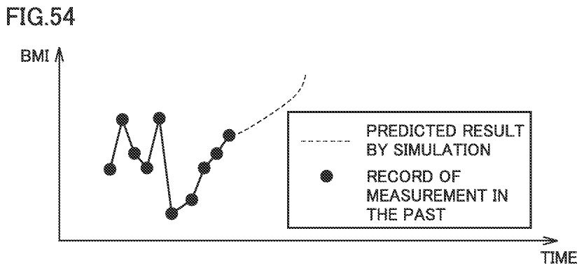

FIG. 54 shows a result of predicting how an indicator changes according to the present embodiment. Referring to FIG. 54, as indicated in this graph by a broken line, a result of prediction of how the indicator of user 10 will change is indicated through simulation.

Returning to FIG. 47, control unit 210 compares the value of the indicator of the current state of user 10 with the result of predicting how the indicator will change to determine whether there is a necessary of intervention for user 10 or praising user 10 (step S172).

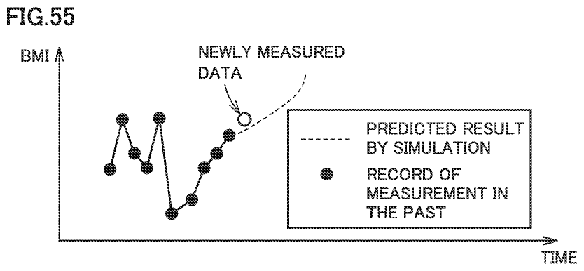

FIG. 55 is a diagram illustrating a comparison between a value of an indicator of the current state and a result of predicting how the indicator will change according to the present embodiment. Referring to FIG. 55, the result of predicting how the indicator will change as shown in FIG. 54 is compared with a new measured value of the indicator of the current state of user 10. In FIG. 55, the new value of the indicator exceeds the predicted result.

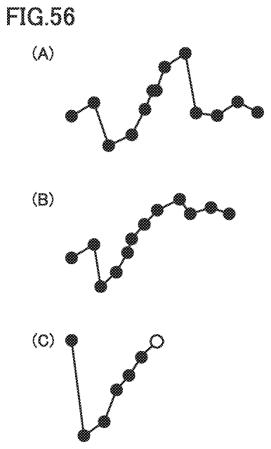

FIG. 56 is a diagram for illustrating a pattern of how an indicator changes according to the present embodiment. How indicators respectively of all users of the system for management by objectives change are accumulated. Of the accumulated information, a point in time series when the indicator proceeds in a bad direction is set as a point of change, and information for a few days before and after the point of change is extracted as a section of change.

Such an extracted section of change is labeled with a feature of the section, such as an improved section or a deteriorated section. Such a section of change thus labelled will be referred to as a changing pattern. Such a changing pattern may be classified in any method, and it may be classified by constructing a feature value, according to a reference for classification, or according to a constructed role.

FIG. 56(A) shows an example of a “still recoverable” changing pattern in which the indicator deteriorates (increases) for several consecutive days (in this example, five consecutive days), and recovers the next day. FIG. 56(B) shows an example of a “base-up” changing pattern in which the indicator deteriorates for several consecutive days (in this example, six consecutive days) and the indicator does not recover the next day or thereafter.

Based on such a changing pattern, it may be determined whether it is necessary to intervene for user 10 or praise user 10. Specifically, of the built changing patterns, one that is the closest to how the indicator's change this time transitions is selected, and from the label attached to the changing pattern, a feature of the change is obtained, and from the change's feature obtained, whether it is necessary to perform intervention for user 10 or praise user 10 is determined.

As shown in FIG. 56(C), when the indicator changes and subsequently attains a value indicated by a white circle, and also deteriorates the next day, it would be the “base-up” changing pattern shown in FIG. 56 (B). Therefore, it is determined that it is necessary to provide intervention for user 10. When user 10 follows this intervention and takes some measure, and the indicator is improved the next day, it will be the “still recoverable” changing pattern shown in FIG. 56(A).

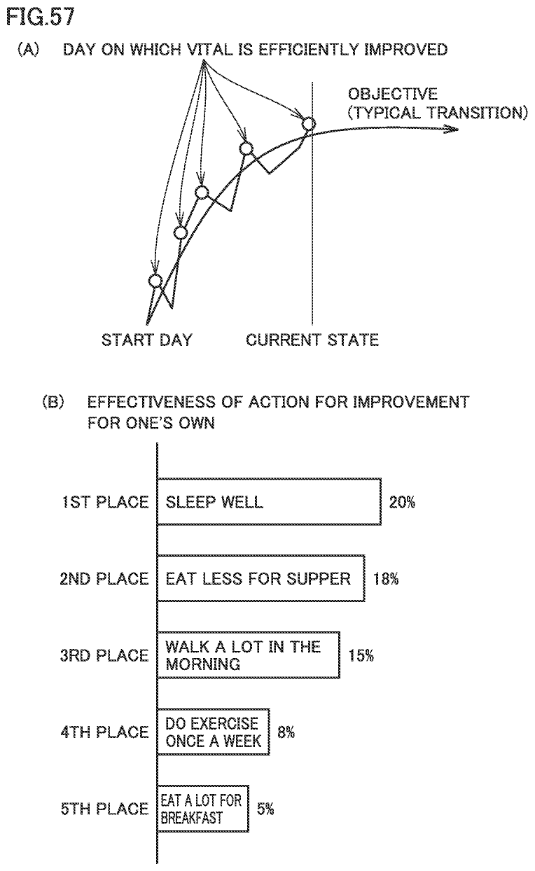

FIG. 57 shows a relationship between a transition of an indicator of an individual and an action of the individual for improvement according to the present embodiment. Referring to FIG. 57, how effective an action taken for improvement by user 10 immediately before a day when user 10 has improved an indicator for an objective, as shown in FIG. 57(A), is, is represented in an order as shown in FIG. 57(B).

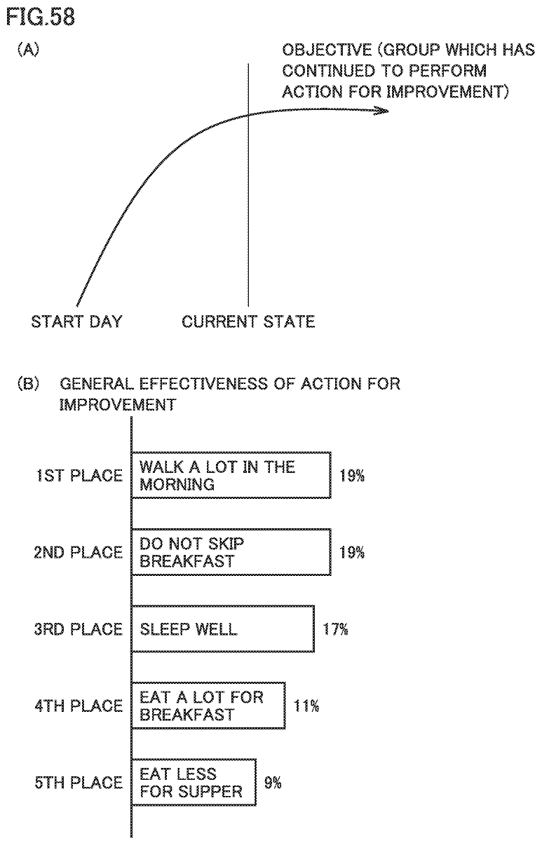

FIG. 58 shows a relationship between a transition of an indicator of a group of users who resemble user 10 that have continued an action for improvement and the action for improvement according to the present embodiment. Referring to FIG. 58, how effective an action that the group of users have taken for improvement and results in an indicator having transitioned, as shown in FIG. 58(A), is, is represented in an order as shown in FIG. 58(B).

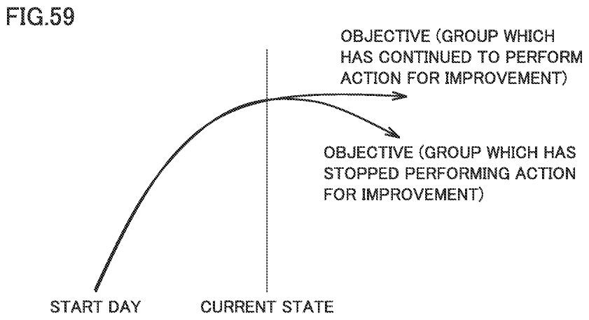

FIG. 59 shows a transition of an indicator of a group of users who resemble user 10 that have stopped an action for improvement according to the present embodiment. Referring to FIG. 59, when stopping the action for improvement is compared with continuing it, the former deteriorates the indicator for the objective.

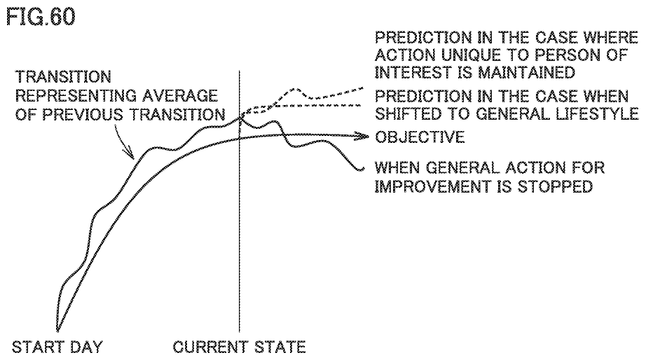

FIG. 60 shows a plurality of patterns of how an indicator transitions in the future according to the present embodiment. Referring to FIG. 60, based on such an analysis as shown in FIGS. 57 to 59, it can be seen that it is effective for user 10 to sleep well, and any action other than that has a small difference in effectiveness from that taken by other users similar to user 10. Based on this analysis, a plurality of patterns for predicting how an indicator will transition in the future, such as shown in FIG. 60, will be created.

Returning to FIG. 47, when it is determined that there is a need for intervention or praise (YES in step S172), control unit 210 creates necessary advice for user 10 (step S173). For example, advice previously stored in storage unit 220 for a situation similar to that of user 10 may be read. Further, a plurality of patterns for predicting how an indicator will transition in the future and their respective explanatory texts, such as shown in FIG. 60, may be created.

Subsequently, control unit 210 transmits the created advice to information communication terminal 100A of user 10 in order to present it on information communication terminal 100A of user 10 (step S174).

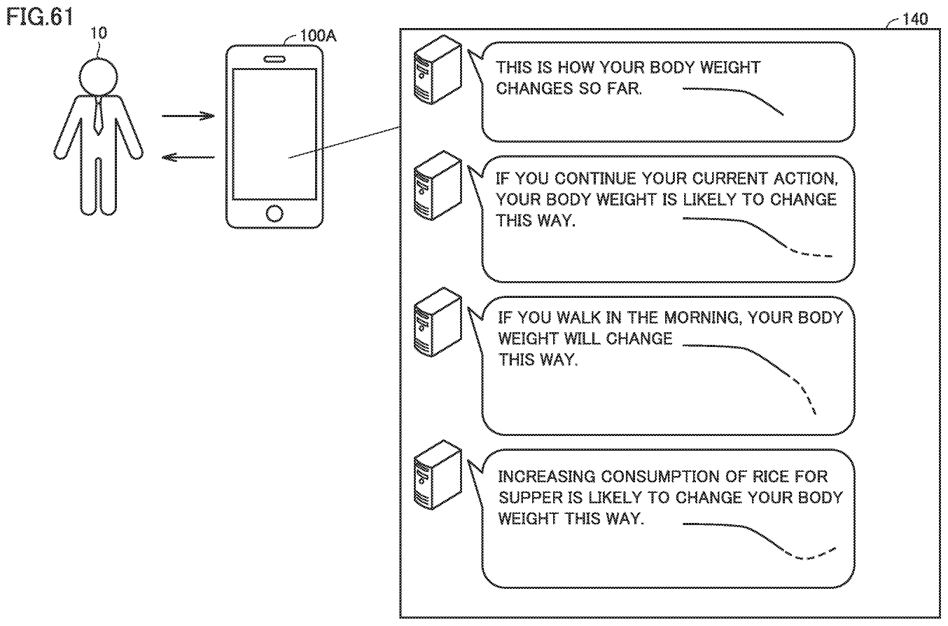

FIG. 61 shows an example of a display screen displayed at output unit 140 of information communication terminal 100A in an objective maintenance process according to the present embodiment. Referring to FIG. 61, these pieces of advice are displayed in step S174 of FIG. 47.

In this way, when a change in an indicator such as a body composition unique to user 10 obtained from a past change so far is controlled and when it is controlled well user 10 is praised, and a sign indicating that the indicator proceeds in a bad direction is caught and advice to prevent that is given to user 10.

Predicting how an indicator's change transitions, as shown in FIGS. 47 to 61, may be applied not only after an objective is achieved but also before the objective is achieved.

### Effects of Embodiment

According to the present embodiment described above, the following effects can be obtained.

(1-1) As shown in step S111 in FIG. 15, in the system for management by objectives, control unit 110 of information communication terminal 100A receives an input of a qualitative first objective for the body of user 10. As shown in steps S112 to S117, control unit 210 of server 200 for management by objectives specifies a quantitative second objective for the body of user 10 from the received first objective. As shown in step S118 and FIG. 28, control unit 110 presents the second objective specified by server 200.

A quantitative objective for the body can thus be indicated without receiving an input of a quantitative numerical objective for the body.

(1-2) As shown in steps S112 to S117 in FIG. 15, control unit 210 converts the first objective into a quantitative objective for at least one of a plurality of feature values for the body to specify a second objective including at least one converted objective. A quantitative objective for a feature value for the body can thus be indicated without receiving an input of a quantitative numerical objective for the body.

(1-3) As shown in steps S112 to S117 in FIG. 15, control unit 210 converts the first objective into a quantitative objective for at least one feature value corresponding to a meaning of the first objective through a linguistic analysis. A quantitative objective for the feature value corresponding to the meaning of the first objective can thus be indicated.

(1-4) As shown in steps S112 to S117 in FIG. 15, the quantitative objective is a range or value included in the range of the feature value corresponding to the meaning of the first objective through the linguistic analysis. A quantitative objective for the feature value corresponding to the meaning of the first objective can thus be indicated.

(1-5) When the first objective has a plurality of meanings through the linguistic analysis, the quantitative objective is a range or value included in the range of the value of the feature value for each meaning. Quantitative objectives for a plurality of feature values corresponding to the meanings of the first objective can thus be indicated.

(1-6) As shown in steps S112 to S117 in FIG. 15, when the first objective through the linguistic analysis has a meaning corresponding to a plurality of feature values, and in a multidimensional space having each feature value as an axis the feature value of a meaning has a range overlapping that of the feature value of another meaning, the quantitative objective is a value or range of each feature value corresponding to a position or range in the multidimensional space that is included in the overlapping range. A quantitative objective that satisfies all of a plurality of feature values corresponding to the meaning of the first objective can thus be indicated.

(2-1) As shown in steps S121 and S122 in FIG. 29, in the system for management by objectives, control unit 110 of information communication terminal 100A obtains the value of the current state of a prescribed indicator for the body of user 10, an objective value, and a deadline for achieving an objective. As shown in step S127 and FIG. 34, storage unit 220 of server 200 for management by objectives previously associates information indicating tendencies of objective achievement with a plurality of people's attributes and thus stores them, the tendencies being indicated by combination of routes representing transitions of values of prescribed indicators to objective values for achieving objectives for the bodies of the plurality of people and an objective achievement rate for each route. As shown in steps S123 to S128, control unit 210 of server 200 uses the tendencies indicated by the information stored in storage unit 220 to create a route having a higher objective achievement rate than another route from the value of the current state, objective value and deadline for achieving the objective that are obtained. As shown in step S129 and FIG. 35, control unit 110 presents the route created by server 200. A route suitable for reaching an objective for a body can thus be presented.

(2-2) As shown in FIG. 34, storage unit 220 further stores past objectives for the plurality of people's bodies in association with tendencies. As shown in steps S123 to S128 in FIG. 29, control unit 210 creates a route using a tendency indicated by information stored in a storage unit that is information of any person setting an objective approximate to that of user 10. A route suitable for reaching an objective for a body can thus be presented based on information of another user.

(2-3) As shown in steps S123 to S128 of FIG. 29, control unit 210 creates a route by using a tendency indicated by information stored in storage unit 220 that is information of any person having an attribute approximate to that of user 10. A route suitable for reaching an objective for a body can be presented based on information of another user.

(2-4) As shown in FIG. 34, storage unit 220 stores an objective achievement rate as the tendency. As shown in steps S123 to S128 in FIG. 29, server 200 creates a route by using an objective achievement rate indicated by information stored in storage unit 220 that is information of any person having an attribute approximate to that of user 10. A route suitable for reaching an objective for a body can thus be presented based on information of another user.

(3-1) As shown in steps S141 to S147 of FIG. 37, in the system for management by objectives, control unit 210 of server 200 for management by objectives calculates a degree of divergence of the value of the current state of a prescribed indicator for achieving an objective for the body of user 10 with respect to a route representing a transition of a value of the prescribed indicator to an objective value of the prescribed indicator. As shown in steps S148 to S152, control unit 210 determines an intervention method according to the calculated degree of divergence. As shown in step S155, control unit 210 performs a process for intervening for user 10 in the determined intervention method. This can effectively encourage user 10 for improvement for achieving an objective for his/her body.

(3-2) As shown in steps S151 and S152 of FIG. 37, control unit 210 determines an intervener for user 10 as an intervention method. As shown in step S155, control unit 210 performs a process for urging the determined intervener to perform intervention as a process for performing intervention for user 10. User 10 can thus be effectively encouraged by the determined intervener for improvement for achieving an objective for the body of user 10.

(3-3) As shown in FIG. 38 and FIG. 42, storage unit 220 previously stores information for determination for determining a statistically effective intervention method. As shown in steps S151 and S152 of FIG. 37, control unit 210 uses the information stored in the storage unit for determination to determine an intervention method that is statistically effective for user 10. An intervention method that is statistically effective for user 10 can thus be used to effectively encourage user 10 for improvement for achieving an objective for his/her body.

(3-4) As shown in FIG. 42, storage unit 220 previously associates an objective for a plurality of people's body with effective intervention methods and thus stores them as the information for determination. As shown in steps S151 and S152 in FIG. 37, control unit 210 determines an intervention method based on an intervention method stored in storage unit 220 that is a method for intervention for any person having an objective approximate to that of user 10. An intervention method statistically effective for user 10, specifically, a method for intervention for a person having an objective approximate to that of user 10, can be used to effectively encourage user 10 for improvement for achieving an objective for his/her body.

(3-5) As shown in FIG. 38, storage unit 220 previously stores the user's past effective intervention method(s) as the information for determination. As shown in steps S151 and S152 in FIG. 37, control unit 210 determines an intervention method based on an intervention method stored in storage unit 220. An intervention method statistically effective for user 10, specifically, a statistically effective one of past intervention methods for user 10, can be used to effectively encourage user 10 for improvement for achieving an objective for his/her body.

(4-1) As shown in steps S141 to S147 of FIG. 37, in the system for management by objectives, control unit 210 of server 200 for management by objectives determines whether intervention is necessary as the value of the current state of a prescribed indicator for achieving an objective for the body of user 10 diverges from a route representing a transition of a value of the prescribed indicator to an objective value of the prescribed indicator. Storage unit 220 stores a result of intervention for each intervener for the indicator for the objective. As shown in steps S151 and S152, when control unit 210 determines that intervention is necessary, control unit 210 uses a result of intervention stored in storage unit 220 to determine an intervener for user 10 that has a high degree of contribution to achievement of an objective. As shown in step S155, control unit 210 performs a process for urging the determined intervener to provide intervention. This can effectively encourage user 10 for improvement for achieving an objective for his/her body. Further, this also allows an intervener having a high degree of contribution to achievement of an objective based on a result of intervention to effectively encourage user 10 for improvement for achieving an objective for his/her body.

(4-2) Storage unit 220 stores a result of intervention for each timing thereof for the objective. As illustrated in step S154 of FIG. 37, control unit 210 uses a result of intervention stored in storage unit 220 to determine a timing which has a high degree of contribution to achievement of an objective. An execution unit performs a process for urging an intervener to intervene as timed as determined by a determination unit. User 10 can be encouraged, as timed for a high degree of contribution to achievement of an objective, for improvement for achieving an objective for his/her body.

(4-3) As shown in step S154 of FIG. 37, control unit 210 determines a person who has a high degree of contribution according to a daily habit of user 10. User 10 can be encouraged, by a person who has a high degree of contribution according to a daily habit of user 10, for improvement for achieving an objective for his/her body.

(5-1) As shown in FIGS. 48 and 50, in the system for management by objectives, storage unit 220 of server 200 for management by objectives previously stores a value indicating a change in a value for a body with respect to an action in type or amount. As shown in steps S161 to S171 in FIG. 47, control unit 210 of server 200 uses a value indicating a change stored in storage unit 220 to predict a value indicating a change in a value for the body of user 10 for each action of user 10. As shown in step S174 and FIG. 61, information communication terminal 100A presents a value indicating a change predicted by server 200. A value indicating a change in a value for the body of user 10 for each action of user 10 can thus be predicted.

(5-2) As shown in FIGS. 57 to 60, control unit 210 predicts a value indicating a change caused when a prescribed action is taken and a value indicating a change caused when the prescribed action is not taken. A value indicating a change in a value for the body of user 10 for each action of user 10 when the prescribed action is taken by user 10 and a value indicating a change in the value for the body of user 10 for each action of user 10 when the prescribed action is not taken by user 10 can thus be predicted.

(5-3) As shown in FIG. 50, storage unit 220 previously stores a value indicating a change in a value for the body of user 10 with respect to the user's action in type or amount. A value indicating a change in a value for the body of user 10 for each action of user 10 can be predicted based on a value indicating a change in the value for the body of user 10 with respect to the user's action in type or amount.

(5-4) As shown in FIG. 48, storage unit 220 previously stores values indicating how values for the bodies of a plurality of people change with respect to their actions in type or amount. A value indicating how a value for the body of user 10 will change with respect to each action of user 10 can be predicted based on values indicating how values for the bodies of a plurality of people change with respect to their actions in type or amount.

[Modification]

(1) In the above-described embodiment, a system for management by objectives has been disclosed. However, this is not exclusive, and the disclosure can be regarded as server 200 and information communication terminal 100 for management by objectives included in the system for management by objectives. In addition, the disclosure can be regarded as a program run by server 200 and information communication terminal 100 and a method for management by objectives performed thereby.

Further, the disclosure can be regarded as a computer-readable storage medium having the program stored therein/thereon. This storage medium may be a magnetic tape, a flexible disk, a hard disk or a similar magnetic disk, a CD-ROM, a CD-R, a CD-RW, a DVD-ROM, a DVD-R, a DVD-RW, a DVD-RAM, a DVD+R, a DVD+RW or a similar optical disk, an MO or a similar magneto-optical disk, a memory card, or a USB memory or a similar medium carrying a program in a fixed manner, or may be a medium which carries a program fluidly so as to download a program from a server of an ASP (an application service provider) via a communication network.

(2) In the embodiment described above, server 200 for management by objectives is a single computer. However, this is not exclusive, and server 200 may be a server group composed of a plurality of computers.

(3) In the embodiment described above, a function performed by the system for management by objectives is implemented by a CPU of control unit 210 causing software that is processing of a program described with reference to FIGS. 14, 15, 29, 37, and 47 to run. However, this is not exclusive, and, these functions may partially or entirely be implemented by dedicated hardware.

(4) In the embodiment described above, a part of a function performed by server 200 may be performed by information communication terminal 100. For example, when control unit 210 of server 200 specifies a prescribed value by using prescribed data stored in storage unit 220 and transmits the specified prescribed value to information communication terminal 100, control unit 210 of server 200 may transmit prescribed data stored in storage unit 220 to information communication terminal 100, and control unit 110 of information communication terminal 100 may use the received prescribed data to specify the prescribed value.

(5) The techniques described in the embodiments and the modifications are also intended to be implemented each alone or in combination as much as possible.

The presently disclosed embodiments are to be considered in all respects as illustrative and not restrictive. The scope of the present disclosure is defined by the terms of the claims, rather than the description of the embodiments, and is intended to include any modifications within the scope and meaning equivalent to the terms of the claims.

### REFERENCE SIGNS LIST

10, 20, 30 user, 100, 100A, 100B, 100C information communication terminal, 110, 210, 510 control unit, 120, 220, 520 storage unit, 130, 530 operation unit, 140, 540 output unit, 150, 250 external storage device, 151, 251 storage medium, 160, 170, 570 wireless communication unit, 200, 300 server, 260 communication unit, 500 measuring device, 580 measurement unit, 800, 800A, 800B communication facilities, 900 communication network.

What is claimed is:

1. A system for management by objectives that performs management by objectives for a body of a user, comprising:
a storage unit that previously stores a value indicating a change in a value for a body with respect to an action in type or amount;
a prediction unit that uses the value stored in the storage unit and indicating the change to predict a value indicating a change in a value for the body of the user for each action of the user; and
a presentation unit that presents the value indicating the change predicted by the prediction unit, wherein
an individual prediction model is created from a value previously stored in the storage unit and indicating a change in the user, and a group prediction model is created from values previously stored in the storage unit and indicating changes in a plurality of users resembling the user, and
when an error of the individual prediction model deviates less from a value for the body of the user in a current state than an error of the group prediction model, the prediction unit applies the individual prediction model to predict a value indicating how the user will change,
whereas when the error of the group prediction model deviates less from the value for the body of the user in the current state than the error of the individual prediction model, the prediction unit applies the group prediction model to predict the value indicating how the user will change.

2. The system according to claim 1, wherein the prediction unit predicts a value indicating a change arising when a prescribed action is performed and a value indicating a change arising when the prescribed action is not performed.

3. The system according to claim 1, wherein the storage unit previously stores a value indicating how the value for the body of the user changes with respect to the user's action in type or amount.

4. The system according to claim 1, wherein the storage unit previously stores values indicating how values for bodies of a plurality of people change with respect to actions of the plurality of people in type or amount.

5. The system according to claim 1, further comprising a server and a terminal device, wherein
the server includes the storage unit and the prediction unit, and
the terminal device includes the presentation unit.

6. A server for management by objectives that performs management by objectives for a body of a user, comprising:
a storage unit that previously stores a value indicating a change in a value for a body with respect to an action in type or amount;
a prediction unit that uses the value stored in the storage unit and indicating the change to predict a value indicating a change in a value for the body of the user for each action of the user; and
a transmission unit that transmits the value predicted by the prediction unit and indicating the change to the terminal device for presentation via the terminal device, wherein
an individual prediction model is created from a value previously stored in the storage unit and indicating a change in the user, and a group prediction model is created from values previously stored in the storage unit and indicating changes in a plurality of users resembling the user, and
when an error of the individual prediction model deviates less from a value for the body of the user in a current state than an error of the group prediction model, the prediction unit applies the individual prediction model to predict a value indicating how the user will change,
whereas when the error of the group prediction model deviates less from the value for the body of the user in the current state than the error of the individual prediction model, the prediction unit applies the group prediction model to predict the value indicating how the user will change.

7. A computer-readable storage medium having a program for management by objectives that is executed in a server that performs management by objectives for a body of a user, the server including a storage unit that previously stores a value indicating a change in a value for a body with respect to an action in type or amount; the program causing the server to perform:
predicting, by using the value stored in the storage unit and indicating the change, a value indicating a change in a value for the body of the user for each action of the user; and
transmitting the value indicating the predicted change to the terminal device for presentation via the terminal device, wherein
an individual prediction model is created from a value previously stored in the storage unit and indicating a change in the user, and a group prediction model is created from values previously stored in the storage unit and indicating changes in a plurality of users resembling the user, and
the predicting includes
when an error of the individual prediction model deviates less from a value for the body of the user in a current state than an error of the group prediction model, applying the individual prediction model to predict a value indicating how the user will change, and
when the error of the group prediction model deviates less from the value for the body of the user in the current state than the error of the individual prediction model, applying the group prediction model to predict the value indicating how the user will change.
